(19) United States
(12) Patent Application Publication
(10) Pub. No.: US 2025/0333681 A1
(43) Pub. Date: Oct. 30, 2025

(54) CELL CULTURE DEVICE AND CULTURE VESSEL

(71) Applicant: Panasonic Intellectual Property Management Co., Ltd., Osaka (JP)

(72) Inventors: Koji OKUMURA, Kyoto (JP); Tohru NAKAGAWA, Osaka (JP); Yukiya USUI, Gifu (JP); Hikaru KATO, Osaka (JP); Masateru DOI, Osaka (JP)

(21) Appl. No.: 19/261,087

(22) Filed: Jul. 7, 2025

Related U.S. Application Data
(63) Continuation of application No. PCT/JP2023/044395, filed on Dec. 12, 2023.

(30) Foreign Application Priority Data
Jan. 12, 2023 (JP) 2023-003333

(51) Int. Cl. C12M 1/42; C12M 1/12; C12M 1/26; C12M 1/34; C12M 3/06

(52) U.S. Cl. CPC C12M 35/06; C12M 25/16; C12M 27/16; C12M 33/14; C12M 41/12

(57) ABSTRACT

A cell culture device includes: a vessel arrangement stand on which a culture vessel is arranged, the culture vessel being a vessel in which a second cell generated from a first cell is cultured, wherein the vessel arrangement stand includes: a stage on which the culture vessel is arranged; and a magnet, and before culturing the second cell, a liquid that includes the first cell to which a magnetic bead that is an example of a magnetic particle is attached is held in the culture vessel.

### CROSS REFERENCE TO RELATED APPLICATIONS

This is a continuation application of PCT International Application No. PCT/JP2023/044395 filed on Dec. 12, 2023, designating the United States of America, which is based on and claims priority of Japanese Patent Application No. 2023-003333 filed on Jan. 12, 2023. The entire disclosures of the above-identified applications, including the specifications, drawings and claims are incorporated herein by reference in their entirety.

### FIELD

The present disclosure relates to a cell culture device and a culture vessel used in the cell culture device.

### BACKGROUND

Stem cells such as induced pluripotent stem cells (IPS cells) and embryonic stem cells (ES cells) are known as pluripotent cells that can be produced from the cells of tissues included in, e.g., human skin, organs, and blood. In particular, iPS cells can be produced using cells derived from the patient to be treated, and then differentiated into the cells of each tissue. Thus, in regenerative medicine, there are expectations for iPS cells to be used as transplant materials in autologous transplants, for which rejection is infrequent. For producing example, when iPS cells from blood, hematopoietic stem cells are extracted from the blood, and the extracted hematopoietic stem cells are infected with a virus by using a viral vector. This makes it possible to produce iPS cells by introducing iPS genes into hematopoietic stem cells. Furthermore, when iPS cells obtained in this way are to be used as transplant materials or the like, the iPS cells are propagated through culturing. Moreover, by inducing differentiation of the propagated iPS cells into T cells, for example, the T cells can be used as, e.g., immune cells such as individualized anti-cancer T cells.

When iPS cells are generated from blood, first it is necessary to separate and extract hematopoietic stem cells from the blood, as described above. In this case, a technique of separating hematopoietic stem cells from blood by means of magnetic force using magnetic beads (magnetic particles) or the like is known. For example, Patent Literature (PTL) 1 discloses a method in which magnetized cells are separated from a cell suspension, e.g., blood.

Furthermore, there is investigation being conducted into, when propagating iPS cells produced from hematopoietic stem cells, performing the propagation by using a cell culture device to automatically culture the iPS cells. In this case, the iPS cells can be propagated by supplying a culture medium to the culture vessel in which the iPS cells are set.

As the cell culture device (automatic culture device), there are mainly two types: an open-type cell culture device and a closed-type cell culture device. In the case of the open-type cell culture device, when culturing the target cells (for example, the iPS cells), an open-type culture vessel such as a plate, or a vessel having an openable/closable lid can be used. On the other hand, in the case of the closed-type cell culture device, when culturing the target cells, a closed-type culture vessel, to which a conduit serving as a flow path is connected, is used.

### CITATION LIST

### Patent Literature

PTL 1: Japanese Unexamined Patent Application Publication (Translation of PCT Application) No. 2013-517763

### SUMMARY

### Technical Problem

However, using the conventional cell culture devices, efficiently culturing the target cells is difficult.

To solve such problems, the present disclosure provides a cell culture device and a culture vessel that are capable of efficiently culturing target cells.

### Solution to Problem

One aspect of the cell culture device according to the present disclosure is a cell culture device that includes: a vessel arrangement stand on which a culture vessel is arranged, the culture vessel being a vessel in which a second cell generated from a first cell is cultured, wherein the vessel arrangement stand includes: a stage on which the culture vessel is arranged; and a magnet, and before culturing the second cell, a liquid that includes the first cell to which a magnetic particle is attached is held in the culture vessel.

One aspect of the culture vessel according to the present disclosure is a culture vessel that is arranged in the above-described cell culture device.

### Advantageous Effects

The present disclosure makes it possible to efficiently culture target cells.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a diagram illustrating the configuration of a cell culture device according to an embodiment.

FIG. 2 is a diagram illustrating the configuration of a culture vessel used in the cell culture device according to the embodiment.

FIG. 3 is a diagram illustrating the configuration of a vessel arrangement stand in the cell culture device according to the embodiment.

FIG. 4 is a diagram illustrating the movement when the vessel arrangement stand oscillates, in the cell culture device according to the embodiment.

FIG. 5 is a diagram for describing the ON/OFF control of magnets in the vessel arrangement stand, in the cell culture device according to the embodiment.

FIG. 6A is a diagram for describing a step of introducing air into a first liquid in a cell culture method according to the embodiment.

FIG. 6B is a diagram for describing a first liquid-supplying step (a cell-supplying step) in the cell culture method according to the embodiment.

FIG. 6C is a diagram for describing a magnetic bead-supplying step in the cell culture method according to the embodiment.

FIG. 6D is a diagram for describing an oscillation step in the magnetic bead-supplying step in the cell culture method according to the embodiment.

FIG. 6E is a diagram for describing the movement of the vessel arrangement stand when magnets are ON, in the magnetic bead-supplying step in the cell culture method according to the embodiment.

FIG. 6F is a diagram for describing a second liquid-discharging step in the cell culture method according to the embodiment.

FIG. 6G is a diagram for describing a buffer solution-supplying step (a cleaning step) in the cell culture method according to the embodiment.

FIG. 6H is a diagram for describing a buffer solution-discharging step in the cell culture method according to the embodiment.

FIG. 6I is a diagram for describing a third liquid-supplying step (a viral vector-supplying step) in the cell culture method according to the embodiment.

FIG. 6J is a diagram for describing a culture medium-supplying step in a culturing step in the cell culture method according to the embodiment.

FIG. 6K is a diagram for describing a residual liquid-eliminating step in the culturing step in the cell culture method according to the embodiment.

FIG. 6L is a diagram for describing a culture medium-circulating step in the culturing step in the cell culture method according to the embodiment.

FIG. 6M is a diagram for describing a culture medium-discharging step during culture medium replacement, in the culturing step in the cell culture method according to the embodiment.

FIG. 6N is a diagram for describing a culture medium-supplying step during the culture medium replacement, in the culturing step in the cell culture method according to the embodiment.

FIG. 60 is a diagram for describing a gas replacement step in the culturing step in the cell culture method according to the embodiment.

FIG. 7 is a diagram illustrating the configuration of a culture vessel according to Variation 1.

FIG. 8 is a diagram for describing a usage example of the culture vessel according to Variation 1.

FIG. 9 is a diagram illustrating the configuration of a culture vessel according to Variation 2.

FIG. 10 is a diagram for describing a usage example of the culture vessel according to Variation 2.

FIG. 11 is a diagram illustrating a first variation of the vessel arrangement stand in the cell culture device according to the embodiment.

FIG. 12 is a diagram illustrating the configuration of the vessel arrangement stand and an example of the culture vessel arranged on the vessel arrangement stand, in the cell culture device according to the embodiment.

FIG. 13 is a diagram illustrating a second variation of the vessel arrangement stand in the cell culture device according to the embodiment.

FIG. 14 is a diagram illustrating the configuration of a culture vessel according to Variation 3.

### DESCRIPTION OF EMBODIMENTS

### Circumstances Leading to the Present Disclosure

First, before describing the embodiments of the present disclosure, the circumstances leading to obtaining one aspect of the present disclosure will be described.

Investigation is being conducted into using a cell culture device to generate target cells, e.g., iPS cells, from a cell suspension, e.g., blood, and propagate the target cells by culturing. For example, when generating iPS cells from blood, hematopoietic stem cells are extracted from the blood, iPS cells are generated from the extracted hematopoietic stem cells, and the generated iPS cells are cultured by using a culture medium. Thus, when generating iPS cells from blood, it is first necessary to extract the hematopoietic stem cells from blood and collect the hematopoietic stem cells. In this case, it is conceivable to extract the hematopoietic stem cells from blood and collect the hematopoietic stem cells using the method disclosed in PTL 1. Specifically, it is conceivable to extract hematopoietic stem cells by magnetizing hematopoietic stem cells included in blood using magnetic beads or the like and attracting the magnetized hematopoietic stem cells using magnetic force, and then collect the hematopoietic stem cells.

However, in the method disclosed in PTL 1, a plurality of magnetic columns are used when extracting the magnetized hematopoietic stem cells. Consequently, the efficiency of collecting the hematopoietic stem cells may be affected by the magnetic columns, leading to a risk of extraction loss (collection loss). Furthermore, since this method involves the use of disposable magnetic columns, the cost increases.

Moreover, when the target cells are iPS cells or the like, after extracting the hematopoietic stem cells, it is necessary to generate iPS cells from the extracted hematopoietic stem cells, and then propagate the generated iPS cells by culturing. Thus, there is an increased risk of the closed system failing and exposure to contaminants occurring.

It is thus difficult, using a single cell culture device, to extract hematopoietic stem cells as first cells from a cell suspension, generate iPS cells as second cells from the hematopoietic stem cells, and then propagate the iPS cells.

Accordingly, as the result of earnest investigation into such a problem, the present inventors have found a method in which first cells (hematopoietic stem cells) are extracted, in a culture vessel for culturing cells, from a cell suspension, second cells (IPS cells) that serve as the targets are generated from the first cells in the culture vessel as is, and continuing from that, the second cells are cultured in the culture vessel.

Specifically, one aspect of the cell culture device according to the present disclosure is a cell culture device that includes: a vessel arrangement stand on which a culture vessel is arranged, the culture vessel being a vessel in which a second cell generated from a first cell is cultured, wherein the vessel arrangement stand includes: a stage on which the culture vessel is arranged; and a magnet, and before culturing the second cell, a liquid that includes the first cell to which a magnetic particle is attached is held in the culture vessel. Thus providing a magnet to the stage on which the culture vessel is arranged makes it possible to, in the culture vessel for culturing the second cells that serve as the targets, extract the first cells from which the second cells are generated. In other words, the extracting of the first cells from the liquid that includes the first cells and the culturing of the second cells obtained from the first cells can be performed using the same culture vessel. This makes it possible to efficiently culture the second cells that serve as the targets.

Furthermore, in one aspect of the cell culture device according to the present disclosure, the cell culture device may include: a pump that discharges a liquid from the culture vessel, wherein when the pump discharges, from the culture vessel, the liquid that includes the first cell to which the magnetic particle is attached, the magnet may apply a magnetic load to the culture vessel to attract the first cell.

This configuration makes it possible to discharge unneeded liquid and selectively retain the first cells in the culture vessel.

Furthermore, in one aspect of the cell culture device according to the present disclosure, the cell culture device may include: an oscillation mechanism that oscillates the stage to cause the stage to tilt with respect to a horizontal direction, wherein the vessel arrangement stand may include a plurality of magnets each of which is the magnet.

This configuration makes it possible to tilt the stage to tilt the culture vessel. This makes it possible to gather the liquid that includes the first cells to a part within the culture vessel, whereby the first cells can be efficiently gathered inside the culture vessel, even when the liquid in the culture vessel is small in amount.

Furthermore, in one aspect of the cell culture device according to the present disclosure, the plurality of magnets may be disposed at a plurality of positions in a direction in which the stage tilts.

This configuration makes it possible to efficiently attract the first cells to the culture vessel by using the plurality of magnets.

Furthermore, in one aspect of the cell culture device according to the present disclosure, the cell culture device may include: a pump that discharges a liquid from the culture vessel, wherein when the pump discharges, from the culture vessel, the liquid that includes the first cell to which the magnetic particle is attached, the first cell to which the magnetic particle is attached may be attracted by using, among the plurality of magnets, at least one magnet that is positioned closer to a fulcrum at which the stage tilts.

This configuration makes it possible to efficiently attract the first cells inside the culture vessel to extract the first cells.

Furthermore, in one aspect of the cell culture device according to the present disclosure, the plurality of magnets may be controlled to attract the second cell by using a greater number of the plurality of magnets than the at least one magnet, the second cell having been cultured.

This configuration makes it possible, even when a large quantity of the second cells have been propagated by the culturing, to easily attract the large quantity of the second cells by using the magnets. This makes it possible to efficiently collect the second cells. Furthermore, in one aspect of the cell culture device according to the present disclosure, a heater may be provided to the stage.

This configuration makes it possible to keep the temperature of the liquid inside the culture vessel at a constant temperature (for example, 37° C.). This makes it possible to more efficiently culture the second cells.

Furthermore, in one aspect of the cell culture device according to the present disclosure, the magnet and the heater may be alternately arranged. In this case, the magnet and the heater may be arranged in a stripe pattern.

This configuration makes it possible to achieve both of keeping the temperature of the liquid inside the culture vessel constant, and attracting the first cells by using the magnets.

Furthermore, in one aspect of the cell culture device according to the present disclosure, the cell culture device may include: a controller that controls output of the heater in accordance with a weight of a liquid in the culture vessel or a tilt of the stage.

This configuration makes it possible to adjust the temperature of the liquid inside the culture vessel to the optimal temperature.

Furthermore, in one aspect of the cell culture device according to the present disclosure, the cell culture device may include: a hold-down plate that holds down, on the stage, the culture vessel arranged on the stage.

This configuration makes it possible to uniformly hold down the culture vessel on the stage, thereby making it possible to more efficiently culture the second cells. Furthermore, since it is possible to uniformly hold down the culture vessel on the magnets, the attraction of the first cells by the magnets can be performed effectively.

Furthermore, in one aspect of the cell culture device according to the present disclosure, the hold-down plate may be held by an elastic body.

The bulging of the culture vessel arranged on the stage changes in accordance with the amount of liquid supplied to the culture vessel, but when the holding plates are held by the elastic bodies, the height positions of the holding plates are automatically adjusted in accordance with the bulging of the culture vessel. This makes it possible to inhibit the hold-down plates from excessively holding down the culture vessel and causing damage to the culture vessel itself or to the liquid inside the culture vessel. In other words, it is possible to hold the culture vessel on the stage in accordance with the bulging of the culture vessel.

Furthermore, in one aspect of the cell culture device according to the present disclosure, the hold-down plate may be provided at a position that opposes the magnet.

This configuration makes it possible to more effectively attract the first cells by the magnets.

Furthermore, in one aspect of the cell culture device according to the present disclosure, the hold-down plate may be constituted from a magnetic material.

Due to this configuration, each hold-down plate functions as a magnetic core, whereby the magnetic flux density passing through the culture vessel can be improved. This makes it possible to more effectively attract the first cells by the magnets.

Furthermore, in one aspect of the cell culture device according to the present disclosure, the magnet may be a permanent magnet, and the cell culture device may include a movement mechanism that is capable of moving the magnet to change a distance between the magnet and the stage.

This configuration makes it possible to change the magnetic force in accordance with the distance between the culture vessel and the magnets. In other words, it is possible to turn the magnetic force applied to the culture vessel ON/OFF without using expensive magnets such as electromagnets. This makes it possible to magnetically attract the first cells with an inexpensive structure. Therefore, a low-cost cell culture device can be realized.

Furthermore, in one aspect of the cell culture device according to the present disclosure, a culture medium and the liquid that includes the first cell may be supplied to the culture vessel, the culture medium being for culturing the second cell.

This configuration makes it possible to perform the extraction of the first cells and the culturing of the second cells obtained from the first cells, using the same culture vessel. This makes it possible to efficiently culture the second cells that serve as the targets. Furthermore, in one aspect of the cell culture device according to the present disclosure, a supply path and a discharge path may be connected to the culture vessel, the supply path being for supplying the culture medium and the liquid that includes the first cell to the culture vessel, the discharge path being for discharging vessel liquid present in the culture vessel, the liquid that includes the first cell may be supplied to the culture vessel from a cell vessel, the culture medium may be supplied to the culture vessel from a culture medium vessel, and the cell vessel, the culture medium vessel, and the culture vessel may define a closed space when the cell vessel and the culture medium vessel are each connected to the supply path and the discharge path.

This configuration makes it possible to, in a closed space, supply the liquid that includes the first cells to the culture vessel, supply the culture medium to the culture vessel, and the like. Furthermore, within the closed space, the vessel liquid present in the culture vessel can be discharged.

Furthermore, in one aspect of the cell culture device according to the present disclosure, a liquid including a viral vector may be supplied to the culture vessel to infect the first cell with a virus to generate the second cell.

This configuration makes it possible to, in the culture vessel, change the first cells into the second cells.

Furthermore, in one aspect of the cell culture device according to the present disclosure, the first cell may be a hematopoietic stem cell, the liquid that includes the first cell may be blood, and the second cell may be an induced pluripotent stem (iPS) cell.

This configuration makes it possible to, in the culture vessel, generate iPS cells from hematopoietic stem cells extracted from blood.

Furthermore, one aspect of the culture vessel according to the present disclosure is a culture vessel that is arranged in the above-described cell culture device. In this case, the culture vessel may include: a supply port through which a culture medium and the liquid that includes the first cell are supplied to the culture vessel, the culture medium being from a culture medium vessel, the liquid that includes the first cell being from a cell vessel; and a discharge port through which vessel liquid present in the culture vessel is discharged, wherein the supply port and the discharge port may be provided separately from each other.

Due to this configuration, the supply port for supplying the culture medium and the liquid that includes the first cells to the culture vessel, and the discharge port for discharging the vessel liquid present in the culture vessel are provided as separate ports. This makes it possible to inhibit the time period when discharging the liquid from the culture vessel (the time of liquid discharge) from becoming longer. Specifically, in the extracting of the first cells, the time period required for discharging the liquid from the culture vessel can be inhibited from becoming longer, and the time period required for discharging the culture medium from the culture vessel for, e.g., culture medium replacement can be inhibited from becoming longer. Furthermore, separating the supply port and the discharge port obviates the need to use the supply port at the time of liquid discharge. Consequently, unneeded residual liquid at the time of liquid discharge does not remain in the supply port. Thus, in the supplying of the next liquid to the culture vessel using the supply port, the unneeded residual liquid also being supplied to the culture vessel can be prevented. Consequently, contamination due to unneeded residual liquid can be prevented from occurring.

These result in making it possible to realize a culture vessel that enables efficiently culturing the second cells that serve as the targets.

Furthermore, in one aspect of the culture vessel according to the present disclosure, the cell vessel, the culture medium vessel, and the culture vessel may define a closed space when the cell vessel and the culture medium vessel are connected to the supply port. This configuration makes it possible to, in a closed space, supply the liquid that includes the first cells to the culture vessel, supply the culture medium to the culture vessel, and the like. Furthermore, within the closed space, the vessel liquid present in the culture vessel can be discharged. This makes it possible to obtain a culture vessel that enables more efficiently culturing the second cells that serve as the targets.

Furthermore, in one aspect of the culture vessel according to the present disclosure, in a top view, a shape of the culture vessel may include two short sides that oppose each other in one direction, and two long sides that oppose each other in an other direction that intersects the one direction, the culture vessel may include: a first supply port and a second supply port that are each the supply port; and a first discharge port and a second discharge port that are each the discharge port, the first supply port may be provided to one of the two short sides of the culture vessel, the first discharge port may be provided to an other of the two short sides of the culture vessel, and the second supply port and the second discharge port may be provided to the other of the two short sides of the culture vessel, or may be separately provided to one side and an other side, respectively, of the two long sides of the culture vessel.

This configuration makes it possible, when circulating the culture medium inside and outside of the culture vessel using the supply port and the discharge port, to select a culture medium circulation route in accordance with the fluid volume of the culture medium in the culture vessel. This makes it possible to efficiently circulate the culture medium. Consequently, it is possible to obtain a culture vessel that enables more efficiently culturing the second cells that serve as the targets.

Furthermore, in one aspect of the culture vessel according to the present disclosure, a supply path may be connected to the supply port, the supply path being for supplying, to the culture vessel, the culture medium and the liquid that includes the first cell, a discharge path may be connected to the discharge port, the discharge path being for discharging the vessel liquid present in the culture vessel, and the supply path and the discharge path may be provided separately from each other.

Due to this configuration, the supply path for supplying the culture medium and the liquid that includes the first cells to the culture vessel, and the discharge path for discharging the vessel liquid present in the culture vessel are connected to the culture vessel as separate flow paths. This makes it possible to inhibit the time period when discharging the liquid from the culture vessel (the time of liquid discharge) from becoming longer. Specifically, in the extracting of the first cells, the time period required for discharging the liquid from the culture vessel can be inhibited from becoming longer, and the time period required for discharging the culture medium from the culture vessel for, e.g., culture medium replacement can be inhibited from becoming longer.

Furthermore, separating the supply path and the discharge path obviates the need to use the supply path at the time of liquid discharge. Consequently, unneeded residual liquid at the time of liquid discharge does not remain in the supply path. Thus, in the supplying of the next liquid to the culture vessel using the supply path, the unneeded residual liquid also being supplied into the culture vessel can be prevented. Consequently, contamination due to unneeded residual liquid can be prevented from occurring.

Furthermore, due to separating the supply path and the discharge path, the flow path of each of the supply path and the discharge path can be designed separately. In other words, an optimal flow path can be designed for each of the supply path and the discharge path. Specifically, it is possible to: design the flow path diameter, etc. of the supply path or the discharge path in accordance with the flow amount of the liquid that flows through the supply path or the liquid that is discharged from the discharge path; design the flow path diameter taking into account only the liquid that flows through the supply path; and the like. This makes it possible to supply liquids such as the culture medium and the liquid that includes the first cells to the culture vessel in an appropriate amount.

As a result of these points, due to separating the supply path and the discharge path, it is possible to realize a culture vessel that enables efficiently culturing the second cells that serve as the targets.

Furthermore, in one aspect of the culture vessel according to the present disclosure, a cross-sectional area of a flow path of the supply path and a cross-sectional area of a flow path of the discharge path may be different from each other.

For example, making the cross-sectional area of the supply path larger than the cross-sectional area of the discharge path makes it possible to efficiently supply, to the culture vessel, liquids such as the culture medium and the liquid that includes the first cells. Furthermore, making the cross-sectional area of the discharge path larger than the cross-sectional area of the supply path makes it possible to efficiently discharge the vessel liquid in the culture vessel.

Furthermore, in one aspect of the culture vessel according to the present disclosure, a filter that traps the second cell may be provided to the discharge path, the second cell being included in the culture medium discharged from the culture vessel, the supply path may include a culture medium supply path through which the culture medium is supplied to the culture vessel, and the culture medium may be supplied to the culture medium supply path to cause the second cell trapped by the filter to return to the culture vessel.

This configuration makes it possible, when the culture medium including the second cells is discharged from the culture vessel, to trap the second cells using the filter and supply the second cells trapped in the filter to the culture vessel, together with the culture medium, when supplying a new culture medium to the culture vessel during culture medium replacement.

Hereinafter, specific exemplary embodiments of the present disclosure are described with reference to the accompanying Drawings. Note that each of the exemplary embodiments described below shows a general or specific example. Thus, the numerical values, shapes, materials, constituent elements, the arrangement and connection of the constituent elements, steps, the processing order of the steps, etc. shown in the following exemplary embodiments are mere examples, and therefore do not limit the scope of the present disclosure. Therefore, among the constituent elements in the following exemplary embodiments, those not recited in any one of the independent claims are described as optional elements.

Furthermore, the respective figures are schematic diagrams and are not necessarily precise illustrations. Furthermore, in the figures, elements which are substantially the same are given the same reference signs, and overlapping description is omitted or simplified.

### Embodiment

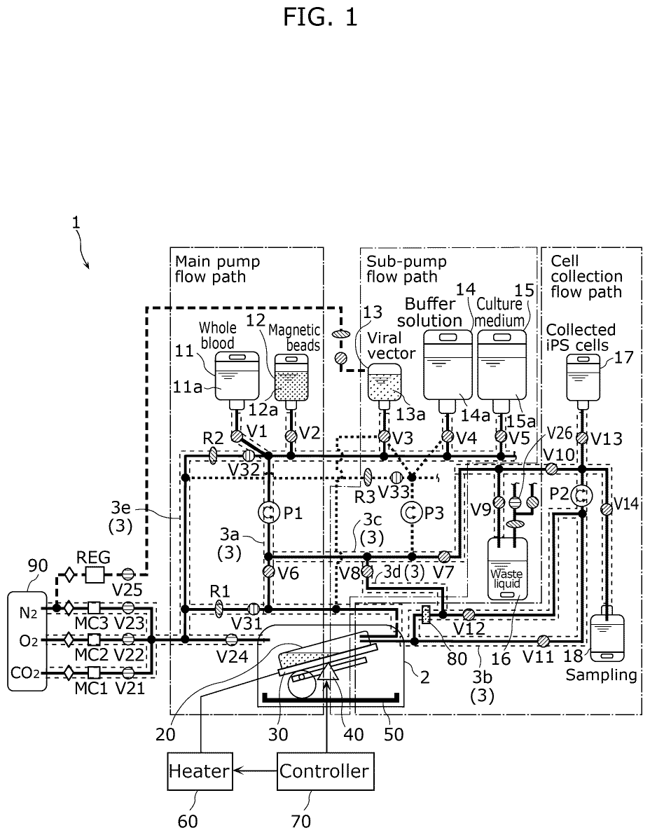

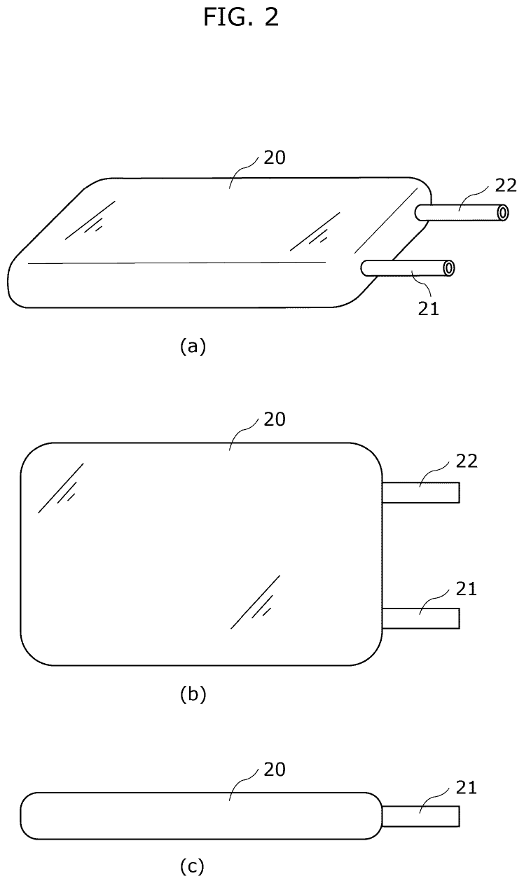

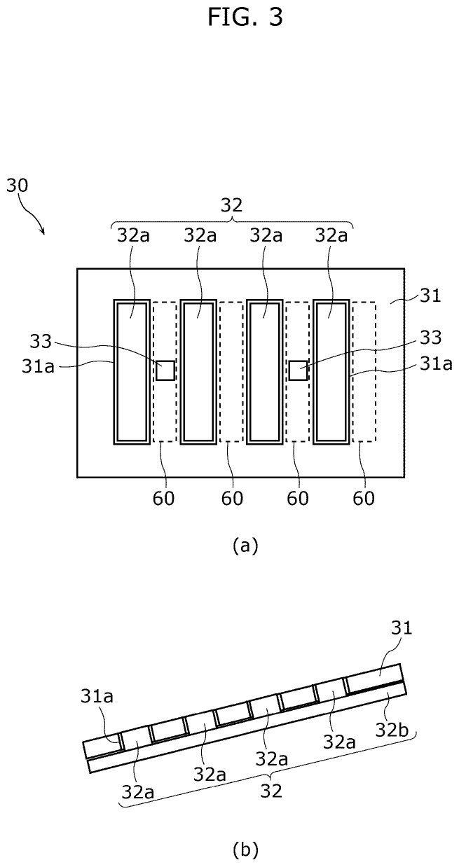

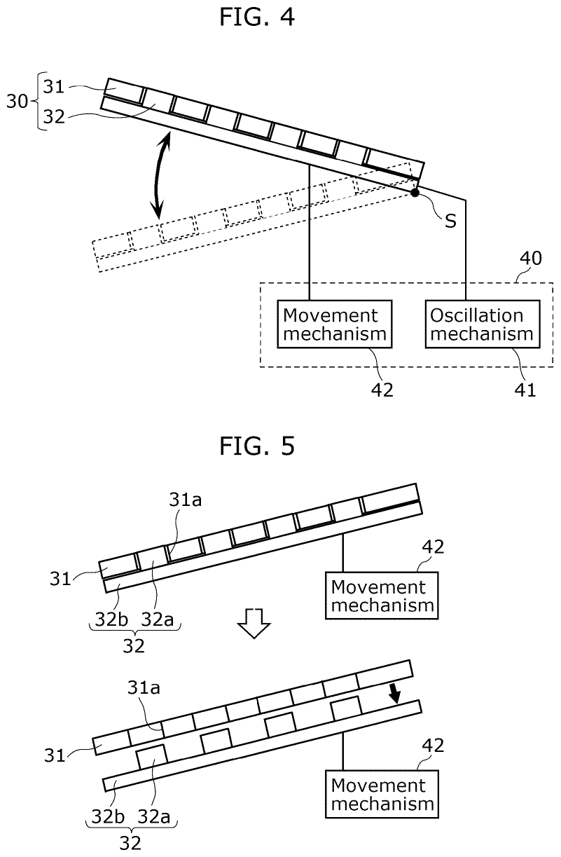

First, the overall configuration of cell culture device 1 according to an embodiment of the present disclosure will be described with reference to FIG. 1 to FIG. 5. FIG. 1 is a diagram illustrating the configuration of cell culture device 1 according to the embodiment. FIG. 2 is a diagram illustrating the configuration of culture vessel 20 used in cell culture device 1 according to the embodiment. In FIG. 2, (a) is an exterior perspective view of culture vessel 20, (b) is a top view of culture vessel 20, and (c) is a side view of culture vessel 20. FIG. 3 is a diagram illustrating the configuration of vessel arrangement stand 30 in cell culture device 1 according to the embodiment. In FIG. 3, (a) is a top view of vessel arrangement stand 30, and (b) is a side view of vessel arrangement stand 30 in a tilted state. FIG. 4 is a diagram illustrating the movement when vessel arrangement stand 30 oscillates, and FIG. 5 is a diagram for describing the ON/OFF control of magnet 32a in vessel arrangement stand 30.

Cell culture device 1 is a device for culturing cells that serve as targets (target cells). Cell culture device 1 is a closed-type cell culture device in which various vessels are connected by flow paths (conduits) or the like to achieve a constant sealed state.

In the present embodiment, the target cells are induced pluripotent stem (iPS) cells. Therefore, cell culture device 1 includes a mechanism for propagating iPS cells through culturing. Furthermore, in the present embodiment, the iPS cells are produced from hematopoietic stem cells included in blood. Specifically, the iPS cells are produced by infecting hematopoietic stem cells extracted from blood with a virus by means of a viral vector, and introducing iPS genes into the hematopoietic stem cells.

Furthermore, cell culture device 1 includes not only the mechanism for culturing iPS cells, but also a mechanism for extracting, from blood, the hematopoietic stem cells from which the iPS cells are generated, and a mechanism for imparting iPS genes to the hematopoietic stem cells. In other words, cell culture device 1 is capable of automatically and continuously performing a series of steps from producing iPS cells from blood to culturing the iPS cells.

Note that the target cells to be cultured by cell culture device 1 are not limited to iPS cells. For example, T cells obtained by inducing further differentiation of the iPS cells cultured by cell culture device 1 may be the target cells. In this case, cell culture device 1 may include a mechanism that is able to induce differentiation of the cultured iPS cells. Furthermore, the target cells may be stem cells other than iPS cells, such as ES cells, or may be cells other than stem cells.

Hereinafter, the specific structure of cell culture device 1 according to the present embodiment will be described in detail.

As illustrated in FIG. 1, cell culture device 1 includes, as various vessels, cell vessel 11, magnetic bead vessel 12, viral vector vessel 13, buffer vessel 14, culture medium vessel 15, waste liquid collection vessel 16, cell collection vessel 17, and sampling vessel 18.

Cell vessel 11, magnetic bead vessel 12, viral vector vessel 13, buffer vessel 14, and culture medium vessel 15 are liquid-holding vessels in which predetermined liquids to be supplied to culture vessel 20 are held. On the other hand, waste liquid collection vessel 16, cell collection vessel 17, and sampling vessel 18 are liquid-collecting vessels that collect liquids that are discharged from culture vessel 20.

These liquid-holding vessels and liquid-collecting vessels are, for example, transparent bags made from transparent resin films, but are not limited thereto. For example, these vessels may be made vessels made of glass or stainless steel.

Cell culture device 1 includes vessel attachment portions for attaching each of these vessels. For example, these vessel attachment portions have structures that allow for hanging and holding vessels. These vessels are replaceable, and can each be attached to a vessel attachment portion or removed from a vessel attachment portion. For example, these vessels may be replaced each time target cells are cultured.

Cell vessel 11 is a vessel that holds first liquid 11a, which is a cell suspension that includes first cells 11b. In the present embodiment, blood is held, as first liquid 11a, in cell vessel 11. First liquid 11a, which is blood, includes at least hematopoietic stem cells as first cells 11b.

Magnetic bead vessel 12 is a vessel that holds second liquid 12a that includes magnetic beads. The magnetic beads (magnet beads) are an example of magnetic particles, and are adsorbed to certain cells included in the cell suspension. In the present embodiment, the magnetic beads included in second liquid 12a have the function of being adsorbed to hematopoietic stem cells included in blood.

Viral vector vessel 13 is a vessel in which third liquid 13a that includes a viral vector is held. The viral vector is a vector that includes a virus used for imparting certain genes to cells. In the present embodiment, the viral vector included in third liquid 13a is used for imparting iPS genes to hematopoietic stem cells included in blood.

Buffer vessel 14 is a vessel that holds buffer solution 14a. In the present embodiment, buffer vessel 14 holds a cleaning solution as buffer solution 14a.

Culture medium vessel 15 is a vessel that holds culture medium 15a for culturing cells. In the present embodiment, since the target cells are iPS cells (second cells), culture medium vessel 15 holds at least culture medium 15a for culturing the iPS cells. Culture medium 15a is a culture solution including, e.g., nutrients necessary for cell growth. Culture medium 15a may be either a natural culture medium or a synthetic culture medium.

Note that in FIG. 1, only one culture medium vessel 15 is illustrated; however, a plurality of culture medium vessels 15 may be provided. For example, in a case in which culture medium 15a in culture vessel 20 is to be replaced, a plurality of culture medium vessels 15 may be provided. In this case, the supply states of each of the plurality of culture medium vessels 15 can be controlled separately by valves.

Waste liquid collection vessel 16 is a vessel for collecting liquid that has become unneeded in cell culture device 1. For example, liquid in culture vessel 20 that has become unneeded is supplied to waste liquid collection vessel 16.

Cell collection vessel 17 is a vessel for collecting target cells. Since in the present embodiment, the target cells are iPS cells, cultured and propagated iPS cells are collected in cell collection vessel 17.

Sampling vessel 18 is a vessel for sampling, e.g., liquids in cell culture device 1. Thus, liquids, etc. serving as the sampling subjects are supplied to sampling vessel 18.

As illustrated in FIG. 1, cell culture device 1 further includes: culture vessel 20, vessel arrangement stand 30, control mechanism 40, weight sensor 50, heater 60, controller 70, filter 80, and gas supply device 90.

Culture vessel 20, vessel arrangement stand 30, control mechanism 40, and weight sensor 50 are provided inside processing chamber 2. Processing chamber 2 is not a sealed space, but may be a closed-type processing chamber that has become a sealed space. Processing chamber 2 has an openable/closable cover that allows culture vessel 20 to be set on vessel arrangement stand 30. Opening the cover of processing chamber 2 allows culture vessel 20 to be set on vessel arrangement stand 30. Culture vessel 20 may be replaced with a new culture vessel 20 after the target cells have been cultured.

Culture vessel 20 is a vessel for culturing cells. Cells and the culture medium for culturing the cells are supplied to culture vessel 20. In other words, the cells and the culture medium are held in culture vessel 20. In the present embodiment, culture vessel 20 is a vessel in which iPS cells (second cells) generated from hematopoietic stem cells (first cells) included in blood are cultured. Thus, at least hematopoietic stem cells, iPS cells, and the culture medium are held in culture vessel 20.

Note that the details will be described later, but in the present embodiment, culture vessel 20 is also used for usages other than culturing target cells. For example, culture vessel 20 is used for extracting cells (in the present embodiment, hematopoietic stem cells) from which the target cells are generated, used for generating target cells (in the present embodiment, iPS cells) from these cells, and the like.

Culture vessel 20 is a closed-type bag. Culture vessel 20 has a pouch portion as the vessel main body. Culture vessel 20 is, for example, a flexible, transparent bag that is made from a transparent resin film, but is not limited thereto. For example, culture vessel 20 may be composed of a resin material other than a transparent resin material, may be composed of a material other than a resin material, or may not be flexible. For example, culture vessel 20 may be a vessel made of glass or stainless steel.

As illustrated in FIG. 2, the vessel main body of culture vessel 20 is in the shape of a thin, substantially rectangular cuboid. Thus, the shape, in a top view, of the vessel main body of culture vessel 20 is substantially rectangular. Note that the vessel main body of culture vessel 20 is not limited to being in the shape of a substantially rectangular cuboid.

Culture vessel 20 has a plurality of ports for, for example, supplying a liquid or a gas to the interior of culture vessel 20, and discharging a liquid or a gas from the inside of culture vessel 20. As illustrated in FIG. 2, in the present embodiment, culture vessel 20 has two ports. Specifically, as the two ports, culture vessel 20 has supply port 21 and discharge port 22. Supply port 21 and discharge port 22 are provided to the vessel main body of culture vessel 20. Supply port 21 is a supply opening for supplying a liquid or a gas to culture vessel 20, and discharge port 22 is a discharge opening for discharging a liquid or a gas from culture vessel 20. As an example, supply port 21 and discharge port 22 are each a long, cylindrical tube made from a hard resin, but supply port 21 and discharge port 22 are not limited thereto. Furthermore, supply port 21 and discharge port 22 are the same as each other in terms of shape and size, but are not limited thereto.

In the present embodiment, supply port 21 and discharge port 22 are provided to the same side of the vessel main body of culture vessel 20. Specifically, supply port 21 and discharge port 22 are provided to one short side, among two opposing short sides of the vessel main body of culture vessel 20.

Culture vessel 20 is arranged on vessel arrangement stand 30 in processing chamber 2. Culture vessel 20 arranged on vessel arrangement stand 30 is connected to a conduit included in flow path 3. Specifically, supply port 21 of culture vessel 20 is connected to an end of supply path 3a of flow path 3, and discharge port 22 of culture vessel 20 is connected to discharge path 3b of flow path 3. Note that when replacing culture vessel 20, supply port 21 and discharge port 22 of culture vessel 20 are removed from flow path 3. Culture vessel 20 is thus configured to be removable from flow path 3.

As illustrated in FIG. 3, vessel arrangement stand 30 has stage 31 on which culture vessel 20 is arranged. The details will be described later, but vessel arrangement stand 30 is tilted by oscillation mechanism 41. In other words, stage 31 tilts.

Vessel arrangement stand 30 has not only stage 31, but also magnet member 32 that has magnet 32a. Thus, vessel arrangement stand 30 is capable of applying a magnetic load to culture vessel 20 arranged on vessel arrangement stand 30.

Magnet member 32 has a plurality of magnets 32a, and plate-shaped support member 32b that supports the plurality of magnets 32a. In the present embodiment, each of the plurality of magnets 32a is a permanent magnet, and applies magnetic force. As an example, as illustrated in (a) in FIG. 3, the plurality of magnets 32a are each in the shape of a rectangular cuboid, and are arranged parallel to each other in the short direction of magnets 32a. In other words, the plurality of magnets 32a are arranged in a stripe pattern. The plurality of magnets 32a are disposed in the direction in which stage 31 tilts.

The plurality of magnets 32a are fixed to a flat surface portion of support member 32b. The materials and shape of support member 32b are not particularly limited as long as support member 32b is capable of supporting the plurality of magnets 32a. Note that not only magnets 32a, but also support member 32b may be a permanent magnet. In other words, the entirety of magnet member 32 may be a permanent magnet.

Through hole 31a is provided to stage 31. In the present embodiment, a plurality of through holes 31a are provided to stage 31. The plurality of through holes 31a each have a rectangular opening shape, and are arranged parallel to each other in the short direction of through holes 31a. In other words, the plurality of through holes 31a are provided in a stripe pattern. As illustrated in (b) in FIG. 3, each of the plurality of magnets 32a of magnet member 32 is inserted through a different one of the plurality of through holes 31a provided to stage 31. That is to say, the plurality of through holes 31a are provided to correspond to the plurality of magnets 32a. Furthermore, heater 60 is provided to stage 31. Specifically, heater 60 is embedded in stage 31. Heater 60 is a heating portion for heating culture vessel 20 arranged on stage 31. Providing heater 60 to stage 31 makes it possible to keep the liquid inside culture vessel 20 arranged on stage 31 at a constant temperature (for example, 37° C.). Heater 60 is, for example, a cassette heater, but is not limited thereto.

In the present embodiment, a plurality of heaters 60 are provided to stage 31. As illustrated in (a) in FIG. 3, each heater 60 is provided between two mutually adjacent through holes 31a. Specifically, heaters 60 and through holes 31a are arranged alternately. Thus, magnets 32a inserted into through holes 31a, and heaters 60 are arranged alternately. The plurality of heaters 60 are, similarly to the plurality of through holes 31a, provided in a stripe pattern. Thus, magnets 32a and heaters 60 are arranged in a stripe pattern with respect to each other. Due to this configuration, it is possible to easily achieve both of: keeping the liquid inside culture vessel 20 arranged on stage 31 at a constant temperature; and attracting the magnetized first cells 11b (hematopoietic stem cells) with magnets 32a.

Turning heaters 60 ON/OFF, and the output of heaters 60 can be controlled by controller 70. Controlling the output of heaters 60 by controller 70 makes it possible to adjust the heating temperature applied to culture vessel 20 by heaters 60.

Furthermore, as illustrated in FIG. 3, temperature sensor 33 is provided to stage 31. Temperature sensor 33 measures the temperature of the vessel liquid present inside culture vessel 20.

Thus, temperature sensor 33 may be arranged in the vicinity of culture vessel 20 when culture vessel 20 is arranged on stage 31. Specifically, temperature sensor 33 may be arranged so as to come in contact with culture vessel 20 arranged on stage 31.

Temperature sensor 33 is positioned on a side of stage 31 that is lower when stage 31 is tilted. In other words, in a top view, temperature sensor 33 may be provided at a position that overlaps with a position at which the vessel liquid collects inside culture vessel 20 when stage 31 is tilted. This makes it possible to accurately measure the temperature of the liquid inside culture vessel 20, even when the position of the liquid surface inside culture vessel 20 changes when stage 31 is oscillated by oscillation mechanism 41, to be described later. In this case, since stage 31 is able to tilt in both the left and right directions, as illustrated in FIG. 3, temperature sensors 33 may be provided at positions that respectively overlap with each of both ends, in the long direction, of culture vessel 20. This makes it possible to measure the temperature of the vessel liquid inside culture vessel 20, whether stage 31 is tilted to the left or to the right.

Note that temperature sensor 33 need not be arranged in the vicinity of culture vessel 20. In this case, temperature sensor 33 can estimate the temperature of the vessel liquid inside culture vessel 20 by measuring the surface temperature of stage 31.

As illustrated in FIG. 4, control mechanism 40 has oscillation mechanism 41 and movement mechanism 42. Control mechanism 40 is controlled by controller 70. Therefore, oscillation mechanism 41 and movement mechanism 42 are controlled by controller 70. Oscillation mechanism 41 and movement mechanism 42 include, for example, a crank mechanism, a link mechanism, and an actuator such as a motor.

Oscillation mechanism 41 has a structure that makes it possible to oscillate vessel arrangement stand 30. Oscillating vessel arrangement stand 30 by using oscillation mechanism 41 makes it possible to oscillate stage 31. In the present embodiment, oscillation mechanism 41 oscillates vessel arrangement stand 30 by changing the tilt of vessel arrangement stand 30.

Thus, oscillation mechanism 41 also has a structure in which the tilt of vessel arrangement stand 30 (stage 31) is changed. As illustrated in FIG. 4, oscillation mechanism 41 can tilt vessel arrangement stand 30 using point S as a fulcrum. In other words, oscillation mechanism 41 can change the tilt of vessel arrangement stand 30 in the manner of a seesaw. Note that point S not only serves as the fulcrum when vessel arrangement stand 30 is tilted, but also as the fulcrum (oscillation fulcrum) when vessel arrangement stand 30 is oscillated.

As an example, when the tilt angle (angle of tilt) of vessel arrangement stand 30 is defined as 0° when vessel arrangement stand 30 is not tilted, oscillation mechanism 41 can change the tilt of vessel arrangement stand 30 in the range of a tilt angle of ±45° (−45°<tilt angle θ<45°). In this case, the maximum variable range when tilting vessel arrangement stand 30 is 90°.

Furthermore, in the case of oscillating vessel arrangement stand 30 by using oscillation mechanism 41, oscillation mechanism 41 can oscillate vessel arrangement stand 30 by changing the angle of vessel arrangement stand 30 so as to reciprocate vessel arrangement stand 30. In this case, oscillation mechanism 41 may oscillate vessel arrangement stand 30 in a predetermined angular range with respect to the horizontal position (tilt angle of 0°), or vessel arrangement stand 30 may be oscillated in a predetermined angular range with respect to a state in which vessel arrangement stand 30 is tilted. As an example, the angular range when oscillating vessel arrangement stand 30 is, for example, ±3°, ±5°, ±10°, ±16°, or the like. Furthermore, the speed when oscillating vessel arrangement stand 30 for 0.01 is, example, to 1.0 reciprocations/second.

Thus changing the tilt of vessel arrangement stand 30 and oscillating vessel arrangement stand 30 makes it possible to change the tilt of culture vessel 20 arranged on vessel arrangement stand 30 and to oscillate culture vessel 20.

Furthermore, in the present embodiment, vessel arrangement stand 30 can be tilted both left and right. This makes it possible to also tilt culture vessel 20 arranged on vessel arrangement stand 30 both left and right. In other words, in accordance with the tilt of vessel arrangement stand 30, one of the two short sides of culture vessel 20 becomes the lower side (or the upper side), the other of the two short sides of culture vessel 20 becomes the lower side (or the upper side), and so forth. Specifically, it is possible to tilt culture vessel 20 such that the side having supply port 21 and discharge port 22 is the lower side, to tilt culture vessel 20 such that the side having supply port 21 and discharge port 22 is the upper side, and the like.

Note that in the present embodiment, oscillation mechanism 41 tilts and oscillates vessel arrangement stand 30 in an integral manner with stage 31 and magnet member 32, but this is not intended to be limiting. For example, oscillation mechanism 41 may tilt and oscillate only stage 31 of vessel arrangement stand 30.

As illustrated in FIG. 5, movement mechanism 42 can move magnet member 32. Specifically, movement mechanism 42 can move magnet member 32 such that the distance between stage 31 and magnet member 32 can be changed. In other words, movement mechanism 42 can make magnet member 32 closer to stage 31 or farther away from stage 31.

When magnet member 32 is moved, the positions of magnets 32a move, with respect to stage 31. Therefore, movement mechanism 42 can move magnets 32a such that the distance between magnets 32a and stage 31 can be changed. In the present embodiment, magnets 32a of magnet member 32 are inserted into through holes 31a in stage 31. Thus, movement mechanism 42 can insert magnets 32a into through holes 31a in stage 31, and remove magnets 32a from through holes 31a.

Weight sensor 50 can detect the weight of the liquid inside culture vessel 20. Specifically, weight sensor 50 detects the weight of the liquid inside culture vessel 20 by detecting the weight of culture vessel 20 including the liquid. Weight sensor 50 is, for example, arranged on vessel arrangement stand 30, but is not limited thereto.

Controller 70 can control control mechanism 40. Specifically, controller 70 can control oscillation mechanism 41 and movement mechanism 42. For example, controlling oscillation mechanism 41 by controller 70 makes it possible to change the tilt of vessel arrangement stand 30 (stage 31) and oscillate vessel arrangement stand 30 (stage 31).

Furthermore, controller 70 can control movement mechanism 42 to bring magnets 32a closer to stage 31 or move magnets 32a farther away from stage 31. This makes it possible to strengthen or weaken the magnetic force applied to culture vessel 20 arranged on stage 31, thereby making it possible to turn ON/OFF the magnetic load acting on culture vessel 20. In other words, by changing the relative distance between magnets 32a, which are permanent magnets, and stage 31, it is possible to switch between a magnet ON state and a magnet OFF state, with respect to culture vessel 20. Note that the magnet ON state is a state in which a magnetic load is applied to culture vessel 20, and the magnet OFF state is a state in which no magnetic load is applied to culture vessel 20.

Note that in the present embodiment, switching between the magnet ON state and the magnet OFF state was performed by changing the relative distance between stage 31 and magnets 32a, but this is not intended to be limiting. In other words, switching between the magnet ON state and the magnet OFF state may be performed without changing the relative distance between stage 31 and magnets 32a. For example, an electromagnet may be arranged on stage 31, and switching between the magnet ON state and the magnet OFF state may be performed by switching the electromagnet ON/OFF.

Furthermore, controller 70 can control heaters 60. Specifically, controller 70 can control turning heaters 60 ON/OFF, control the ON time of heaters 60, change the output of heaters 60, and the like.

Note that controller 70 is a controller that is integrated into cell culture device 1, but is not limited thereto. For example, controller 70 may be an external device outside of cell culture device 1. In this case, controller 70 may be a controller such as a tablet terminal capable of communicating with cell culture device 1 in a wired or wireless manner.

Culture vessel 20 set on vessel arrangement stand 30 may be connected with the various other vessels (cell vessel 11, magnetic bead vessel 12, viral vector vessel 13, buffer vessel 14, culture medium vessel 15, waste liquid collection vessel 16, cell collection vessel 17, and sampling vessel 18) by flow path 3, illustrated by the thick solid line in FIG. 1. Flow path 3 is a conduit through which a fluid such as a liquid or a gas passes.

Culture vessel 20 set on vessel arrangement stand 30, flow path 3 (supply path 3a, discharge path 3b, connection path 3c, culture medium supply path 3d, etc.), and the other various vessels connected to culture vessel 20 via flow path 3 (cell vessel 11, magnetic bead vessel 12, viral vector vessel 13, buffer vessel 14, culture medium vessel 15, waste liquid collection vessel 16, cell collection vessel 17, and sampling vessel 18) define a closed space, and more specifically define a space that has achieved a sterile state by, e.g., being sealed. However, a slight margin of error that is unintended, such as a decrease in the airtightness of the various vessels, is included in the closed space in the present disclosure.

As an example, flow path 3 is a flexible tube made of silicone, but is not limited thereto. For example, flow path 3 may be a rigid pipe made of a resin or a metal that is not flexible. Furthermore, flow path 3 is constituted from a plurality of tubes. Each of the plurality of tubes can be replaced. For example, used tubes may be replaced each time culture vessel 20 is replaced after culturing target cells.

As illustrated in FIG. 1, flow path 3 includes supply path 3a for supplying liquid to culture vessel 20, and discharge path 3b for discharging vessel liquid present in culture vessel 20. Supply path 3a is connected to supply port 21 of culture vessel 20. Discharge path 3b is connected to discharge port 22 of culture vessel 20. In the present embodiment, supply path 3a and discharge path 3b are provided separately from each other. In other words, supply path 3a and discharge path 3b are configured as separate flow paths.

Supply path 3a is a flow path through which liquid supplied to culture vessel 20 flows. Specifically, first liquid 11a held in cell vessel 11, second liquid 12a held in magnetic bead vessel 12, third liquid 13a held in viral vector vessel 13, buffer solution 14a held in buffer vessel 14, and culture medium 15a held in culture medium vessel 15 flow through supply path 3a. Furthermore, supply path 3a is also a collection path in which, when circulating the culture medium that is inside culture vessel 20, that culture medium is collected.

Discharge path 3b is a flow path through which liquid (vessel liquid) inside culture vessel 20 is discharged. Specifically, discharge path 3b is a flow path through which liquid (vessel liquid) that is inside culture vessel 20 and has become unneeded flows. For example, liquid including liquid (first liquid 11a, second liquid 12a, third liquid 13a, buffer solution 14a, etc.) that has been supplied to culture vessel 20 and that has served its purpose and become unneeded flows through discharge path 3b. Note that in the circulating of the culture medium that is inside culture vessel 20, the culture medium discharged from culture vessel 20 flows through discharge path 3b.

In the present embodiment, the cross-sectional area of the flow path of supply path 3a and the cross-sectional area of the flow path of discharge path 3b are the same. Specifically, the flow path diameter of supply path 3a and the flow path diameter of discharge path 3b are the same. However, the cross-sectional area of the flow path of supply path 3a and the cross-sectional area of the flow path of discharge path 3b may be different from each other. In other words, the flow path diameter of supply path 3a and the flow path diameter of discharge path 3b may be different from each other. In this case, when the viscosity of liquid that is to be supplied to culture vessel 20 (first liquid 11a, second liquid 12a, third liquid 13a, buffer solution 14a, culture medium 15a, etc.) is high, making the flow path diameter of supply path 3a larger than the flow path diameter of discharge path 3b makes it possible to efficiently supply this liquid to culture vessel 20. On the other hand, making the flow path diameter of discharge path 3b larger than the flow path diameter of supply path 3a makes it possible to make the fluid volume of discharged liquid at the time of liquid discharge greater than the fluid volume of supplied liquid at the time of liquid supply, whereby a large amount of vessel liquid that has accumulated in culture vessel 20 can be efficiently discharged.

Flow path 3 further includes connection path 3c, which connects supply path 3a with discharge path 3b. Connection path 3c is used for circulating culture medium 15a that is inside culture vessel 20. Specifically, in the circulating of culture medium 15a that is inside culture vessel 20, culture medium 15a inside culture vessel 20 returns to culture vessel 20 by going through discharge path 3b, connection path 3c, and supply path 3a.

Flow path 3 further includes culture medium supply path 3d that supplies culture medium 15a to culture vessel 20. In other words, culture medium 15a supplied from culture medium vessel 15 flows through culture medium supply path 3d. Note that liquid other than culture medium 15a may flow through culture medium supply path 3d. Furthermore, culture medium supply path 3d may be a part of supply path 3a.

Supply path 3a, discharge path 3b, connection path 3c, and culture medium supply path 3d thus function as liquid supply paths through which liquid flows, but are not limited thereto. Specifically, supply path 3a, discharge path 3b, connection path 3c, and culture medium supply path 3d may function as gas supply paths or gas discharge paths through which gas passes. In other words, liquid or gas may pass through all conduits constituting flow path 3 in cell culture device 1.

Furthermore, flow path 3 includes gas supply path 3e to which gas is supplied by gas supply device 90. Gas supply device 90 has the function of supplying gas to gas supply path 3e. In the present embodiment, only gas flows through gas supply path 3e. The gas supplied to gas supply path 3e is supplied to culture vessel 20.

Gas supply path 3e is directly connected to culture vessel 20. In the present embodiment, gas supply path 3e converges with the liquid supply paths of flow path 3, and with a part of the liquid supply paths serving as a part of gas supply path 3e, gas supply path 3e directly connects with culture vessel 20. Specifically, gas supply path 3e converges with supply path 3a. In other words, gas supply path 3e and supply path 3a are directly connected with each other, a part of supply path 3a becomes a part of gas supply path 3e, and gas supply path 3e directly connects with culture vessel 20. Note that gas supply path 3e converging with the liquid supply paths such as supply path 3a makes it possible for gas supply device 90 to supply gas also to the liquid supply paths such as supply path 3a via gas supply path 3e.

In the present embodiment, gas supply path 3e branches, and includes an upper flow path that connects with culture vessel 20 via pump P1, and a lower flow path that connects with culture vessel 20 without going through pump P1. The upper flow path of gas supply path 3e connects with supply path 3a on a side farther from culture vessel 20 (a side closer to the liquid holding vessels such as cell vessel 11), and the lower flow path of gas supply path 3e connects with supply path 3a on a side closer to culture vessel 20 (a side farther from the liquid holding vessels such as cell vessel 11).

The gas that is supplied to culture vessel 20 via gas supply path 3e is mixed gas including, for example, nitrogen, oxygen, and carbon dioxide. Thus, gas supply device 90 has the function of supplying nitrogen (N₂ gas), the function of supplying oxygen (O₂ gas), and the function of supplying carbon dioxide (CO₂ gas). Specifically, gas supply device 90 includes a nitrogen tank in which nitrogen is held, an oxygen tank in which oxygen is held, and a carbon dioxide tank in which carbon dioxide is held.

Note that gas supply device 90 may not supply a mixed gas in which gases of the three of oxygen, nitrogen, and carbon dioxide are mixed, but may separately supply the gases of each of oxygen, nitrogen, and carbon dioxide, or may supply a mixed gas in which two gases among oxygen, nitrogen, and carbon dioxide are mixed. Gas supply device 90 is controlled by controller 70.

Gas supply device 90 supplying gas in this way to culture vessel 20 makes it possible to maintain the concentration of the carbon dioxide included in the gas in culture vessel 20 at 2% to 10% (preferably 5%). Note that gas supply device 90 may supply gas also to the inside of processing chamber 2. This makes it possible to keep the carbon dioxide concentration in processing chamber 2 constant.

The gas supplied to the inside of culture vessel 20 is discharged from discharge path 3b. In other words, discharge path 3b functions as a gas discharge path. Specifically, one end of discharge path 3b, which is a gas discharge path, is directly connected to discharge port 22 of culture vessel 20. Furthermore, the other end of discharge path 3b, which is a gas discharge path, is openable via waste liquid collection vessel 16. In other words, the other end of discharge path 3b, which is a gas discharge path, can be closed and opened. Opening the other end of discharge path 3b, which is a gas discharge path, causes the gas supplied to the inside of culture vessel 20 to be released into the atmosphere via waste liquid collection vessel 16, by going through discharge path 3b.

As illustrated in FIG. 1, valves V1 to V14, valves V21 to V26, and valves V31 to V33 are provided to flow path 3. Valves V1 to V14, valves V21 to V26, and valves V31 to V33 are opening/closing valves that control the opening/closing of flow path 3. As an example, valves V1 to V14 are pinch valves, valves V21 to V26 are solenoid valves, and valves V31 to V33 are air valves. Controlling these valves makes it possible to pass through or stop liquid or gas at the sites at which the valves are provided to flow path 3.

Valves V1 to V6 are provided to supply path 3a. Valve V1 is provided in the vicinity of the port of cell vessel 11, and supplies first liquid 11a held in cell vessel 11 to supply path 3a, or stops the supplying of first liquid 11a to supply path 3a. Valve V2 is provided in the vicinity of the port of magnetic bead vessel 12, and supplies second liquid 12a held in magnetic bead vessel 12 to supply path 3a, or stops the supplying of second liquid 12a to supply path 3a. Valve V3 is provided in the vicinity of the port of viral vector vessel 13, and supplies third liquid 13a held in viral vector vessel 13 to supply path 3a, or stops the supplying of third liquid 13a to supply path 3a. Valve V4 is provided in the vicinity of the port of buffer vessel 14, and supplies buffer solution 14a held in buffer vessel 14 to supply path 3a, or stops the supplying of buffer solution 14a to supply path 3a. Valve V5 is provided in the vicinity of the port of culture medium vessel 15, and supplies culture medium 15a held in culture medium vessel 15 to supply path 3a, or stops the supplying of culture medium 15a to supply path 3a. Valve V6 is provided to supply path 3a between pump P1 and supply port 21 of culture vessel 20, and supplies liquid to culture vessel 20, or stops the supplying of the liquid to culture vessel 20.

Valve V7 is provided to connection path 3c of flow path 3, and controls the liquid that flows through connection path 3c. In the circulating of the culture medium that is inside culture vessel 20, valve V7 is in an open state, and consequently, the culture medium discharged from culture vessel 20 can be returned to culture vessel 20. Furthermore, valve V8 is provided to culture medium supply path 3d of flow path 3, and controls the liquid that flows through culture medium supply path 3d. In the replacing of the culture medium that is inside culture vessel 20, valve V8 is in an open state, and consequently, a new culture medium can be supplied to culture vessel 20 via filter 80.

Valves V9 to V14 are provided to discharge path 3b. Valve V9 is provided in the vicinity of the port of waste liquid collection vessel 16, and passes waste liquid to waste liquid collection vessel 16, or stops the collection of the waste liquid to waste liquid collection vessel 16. Valve V10 is provided between valve V9 and valve V11 in discharge path 3b. Valve V11 is provided between valve V10 in discharge path 3b and discharge port 22 of culture vessel 20. Valve V11 discharges, from culture vessel 20, liquid or gas that is inside culture vessel 20, or stops the discharging of the liquid or gas that is inside culture vessel 20. Valve V12 is provided between valve V10 and, of discharge path 3b connected to culture vessel 20, a site in branched discharge path 3b at which culture medium supply path 3d is connected. Filter 80 is provided between valve V12 in discharge path 3b, and culture vessel 20. In the exchanging of the culture medium that is in culture vessel 20, filter 80 traps cells that are included in the culture medium discharged from culture vessel 20. Specifically, filter 80 traps iPS cells that are discharged from culture vessel 20. The culture medium is sent from the opposite side of filter 80 to cause the cells trapped by filter 80 to return to culture vessel 20. Valve V13 is provided in the vicinity of the port of cell collection vessel 17, and collects cultured cells into cell collection vessel 17, or stops the collecting of the cells to cell collection vessel 17. Valve V14 is provided in the vicinity of the port of sampling vessel 18, and passes sampling liquid to sampling vessel 18, or stops the collecting of the sampling liquid into sampling vessel 18.

Valves V21 to V24 are provided to gas supply path 3e. Valve V21 supplies carbon dioxide from gas supply device 90 to gas supply path 3e, or stops the supplying of carbon dioxide to gas supply path 3e. Valve V22 supplies oxygen from gas supply device 90 to gas supply path 3e, or stops the supplying of oxygen to gas supply path 3e. Valve V23 supplies nitrogen from gas supply device 90 to gas supply path 3e, or stops the supplying of nitrogen to gas supply path 3e. Valve V24 is provided between gas supply device 90 in gas supply path 3e and processing chamber 2, and supplies a mixed gas of carbon dioxide, oxygen, and nitrogen to processing chamber 2, or stops the supplying of the mixed gas to processing chamber 2.

Valve V31 and valve V32 are also provided to gas supply path 3e. Valve V31 is provided to the lower flow path of supply path 3a, and supplies gas to supply path 3a on a side closer to culture vessel 20, or stops the supplying of the gas to supply path 3a. Valve V32 is provided to the upper flow path of supply path 3a, and supplies gas to supply path 3a on a side closer to the liquid holding vessels such as cell vessel 11, or stops the supplying of the gas to supply path 3a. Note that both valve V31 and valve V32 are positioned on an upstream side with respect to gas supply path 3e.

Furthermore, relief valves R1, R2, and R3 are provided to gas supply path 3e in flow path 3. This makes it possible, in the supplying of gas to gas supply path 3e, to inhibit excess pressure from being applied inside culture vessel 20. Note that relief valve R1 is provided to the lower flow path of gas supply path 3e, and relief valve R2 is provided to the upper flow path of gas supply path 3e.

Furthermore, pumps P1, P2, and P3 are provided to flow path 3. Pumps P1, P2, and P3 each have the function of sucking up or sending liquid or gas that flows through flow path 3. Pump P1 is provided to supply path 3a. Pump P1 controls the flow of liquid or gas that flows through the main pump flow path in flow path 3. Specifically, pump P1 is provided between liquid holding vessels such as cell vessel 11 and culture vessel 20, in supply path 3a.

Furthermore, pump P2 is provided to discharge path 3b. Pump P2 controls the flow of liquid or gas that flows through the cell collection flow path in flow path 3. Specifically, pump P2 is provided between culture vessel 20 and liquid holding vessels such as waste liquid collection vessel 16, in discharge path 3b. Note that pump P3 controls the flow of liquid or gas that flows through the sub-pump flow path in flow path 3.

Furthermore, mass controllers MC1, MC2, and MC3 are provided to flow path 3. Mass controllers MC1, MC2, and MC3 control the flow amount of gas that is supplied to gas supply path 3e from gas supply device 90. Specifically, mass controller MC1 adjusts the flow amount of carbon dioxide gas, mass controller MC2 adjusts the flow amount of oxygen gas, and mass controller MC3 adjusts the flow amount of nitrogen gas. Note that regulator REG is also provided to flow path 3.

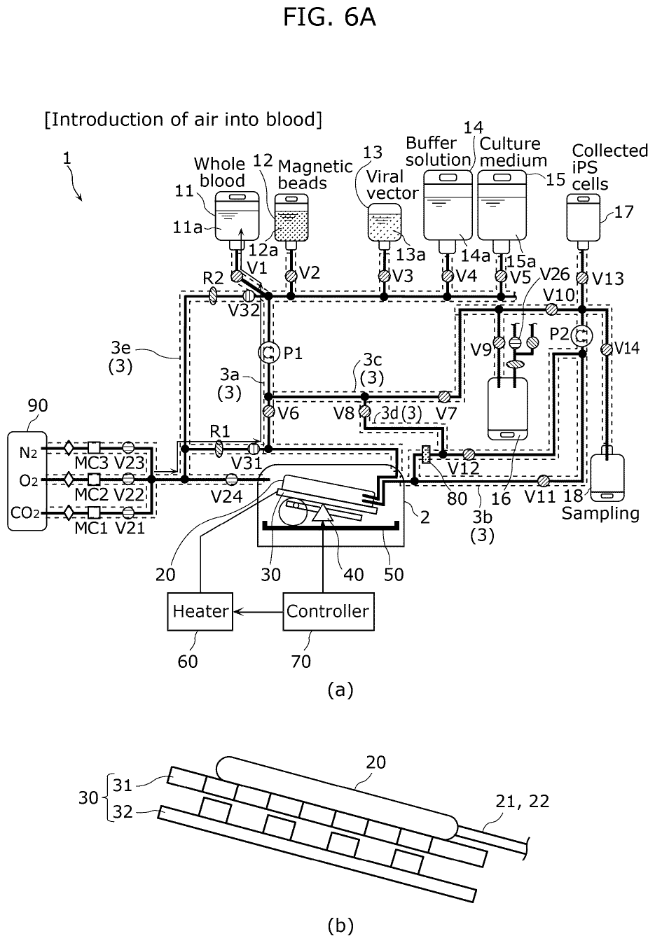

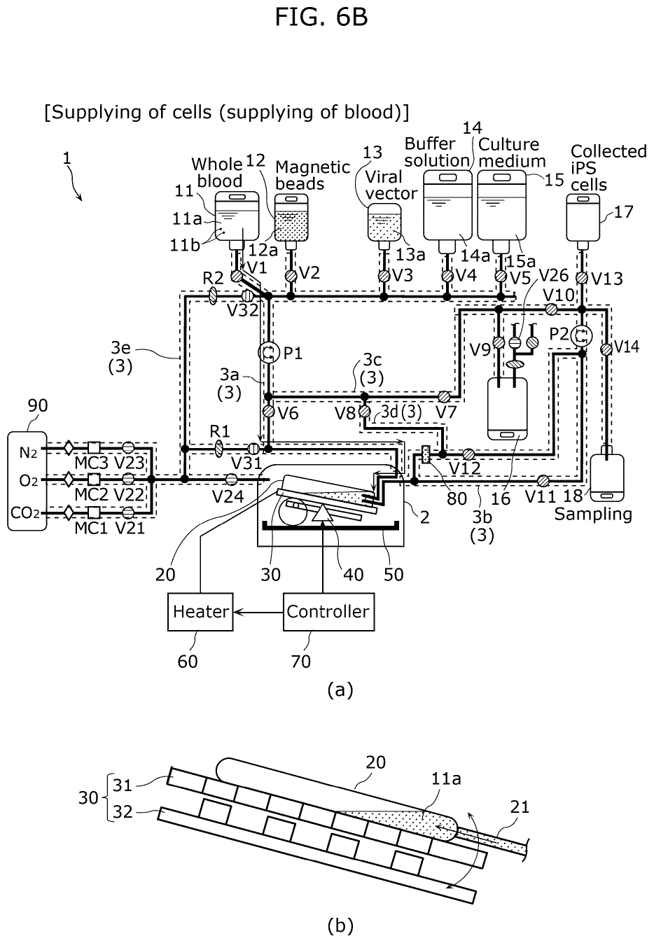

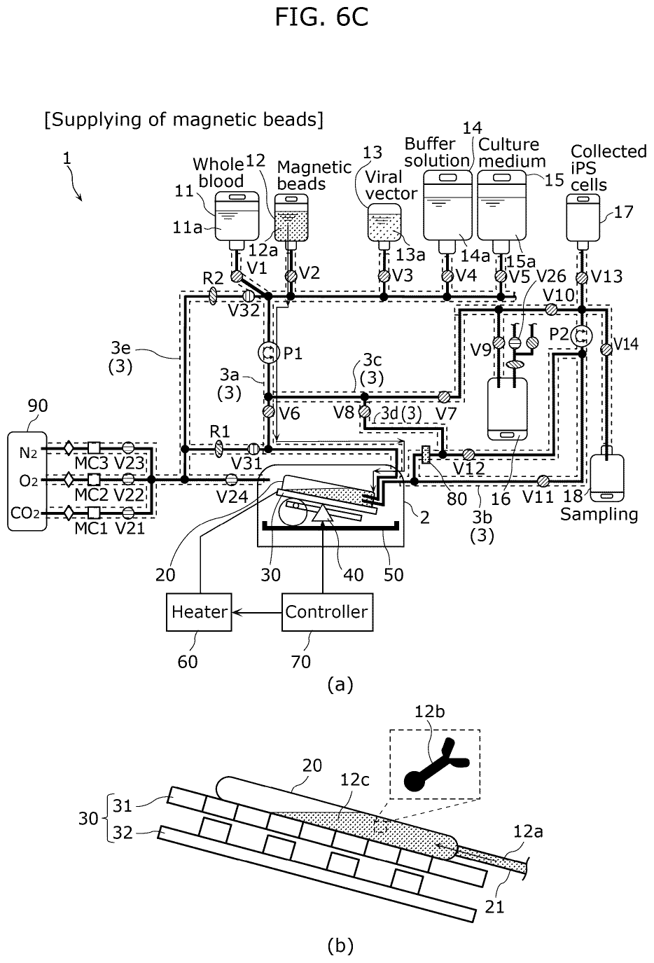

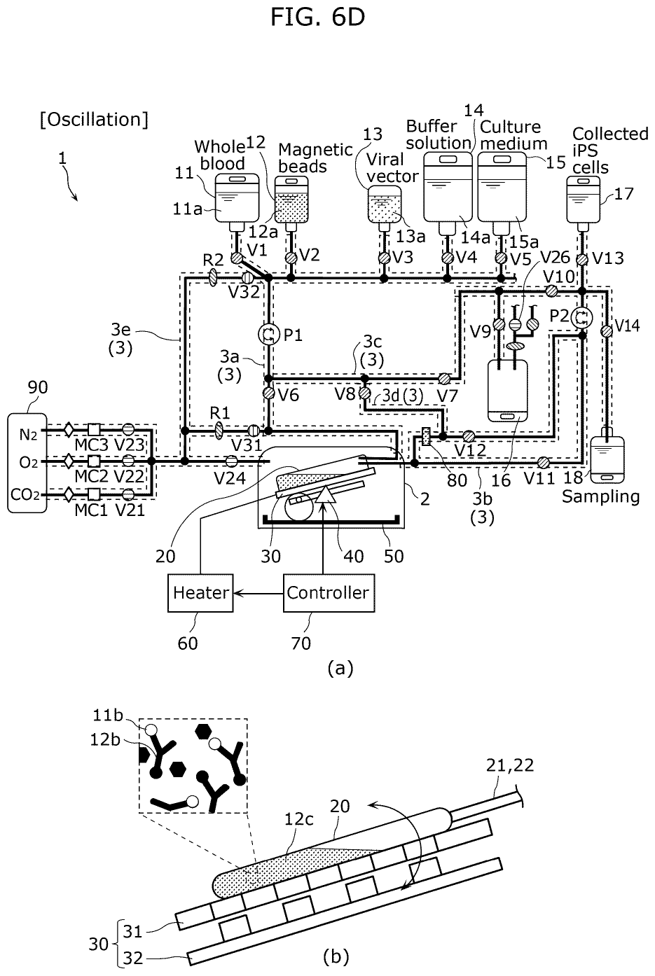

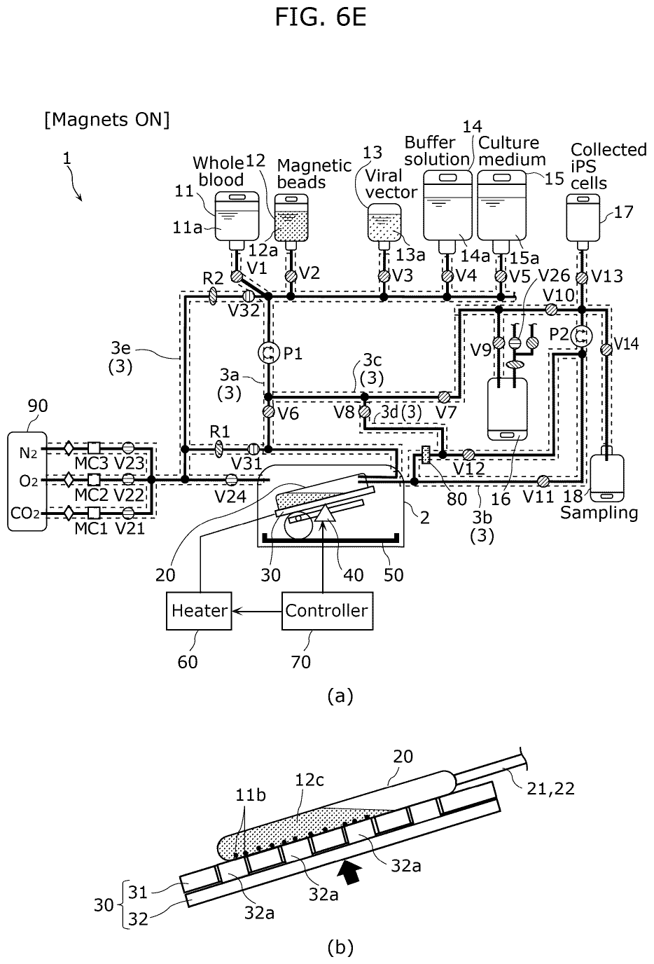

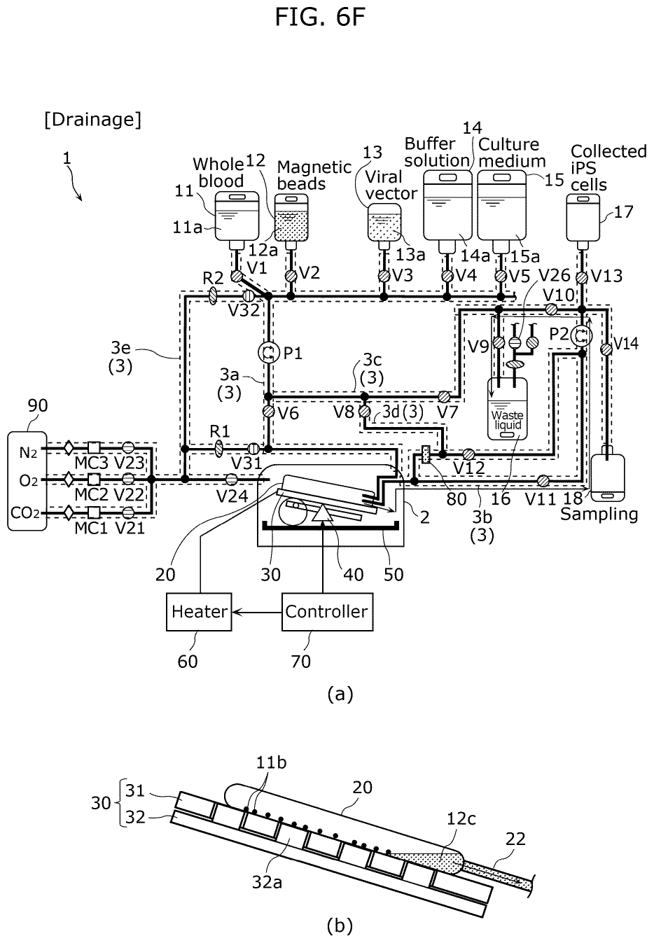

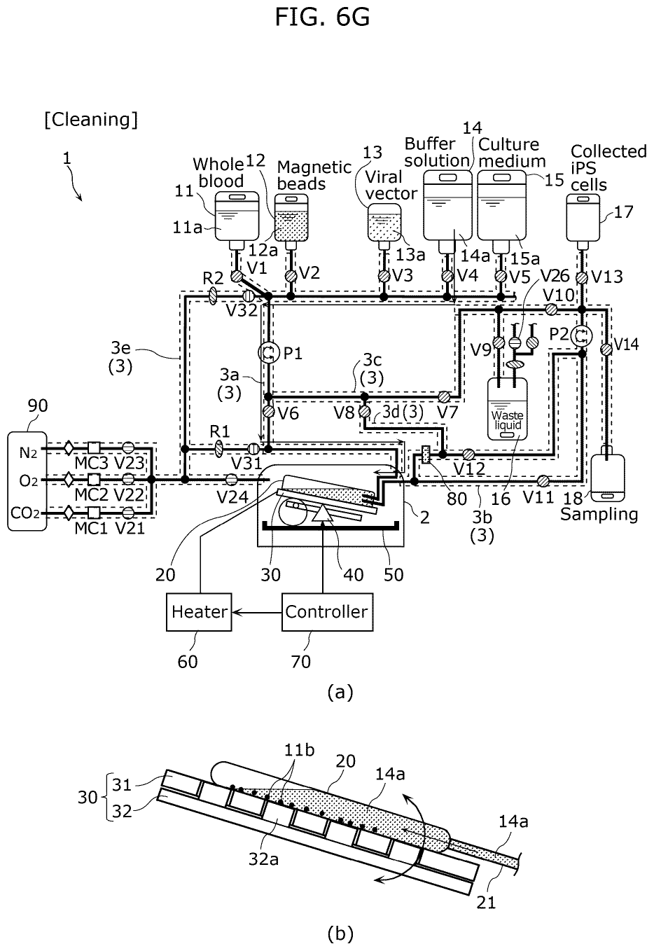

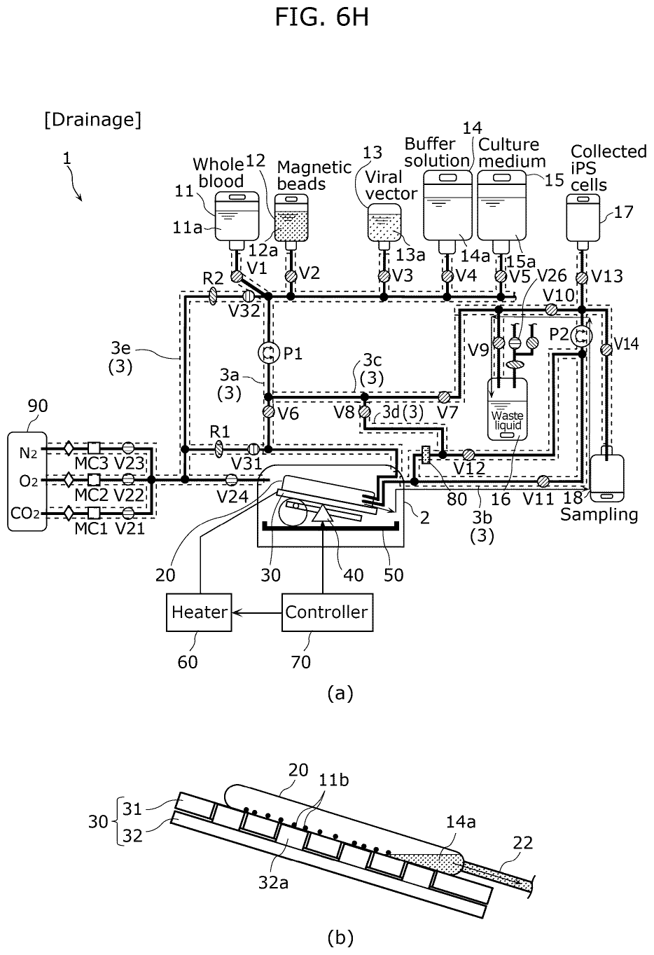

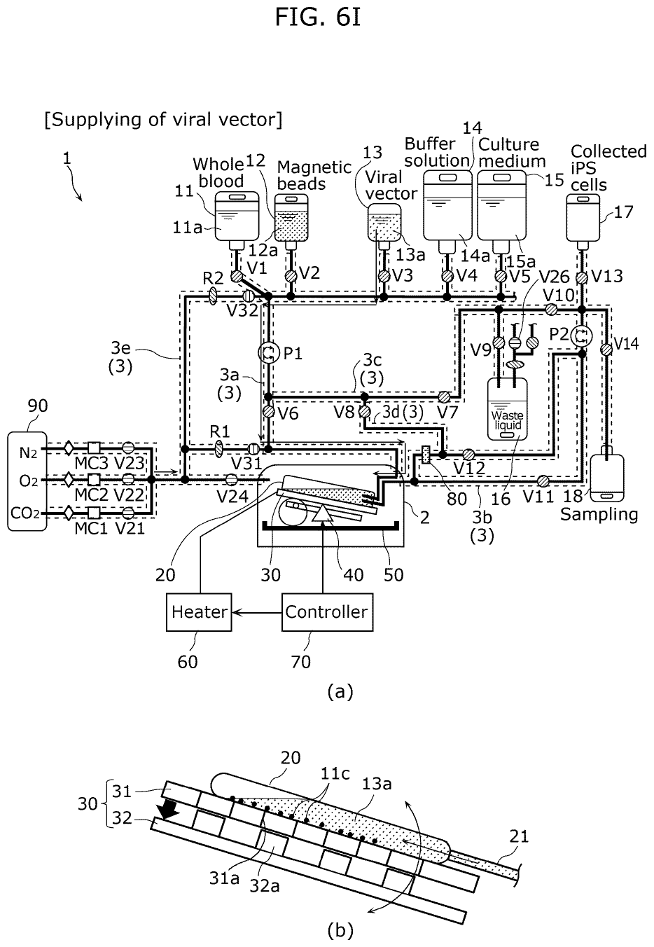

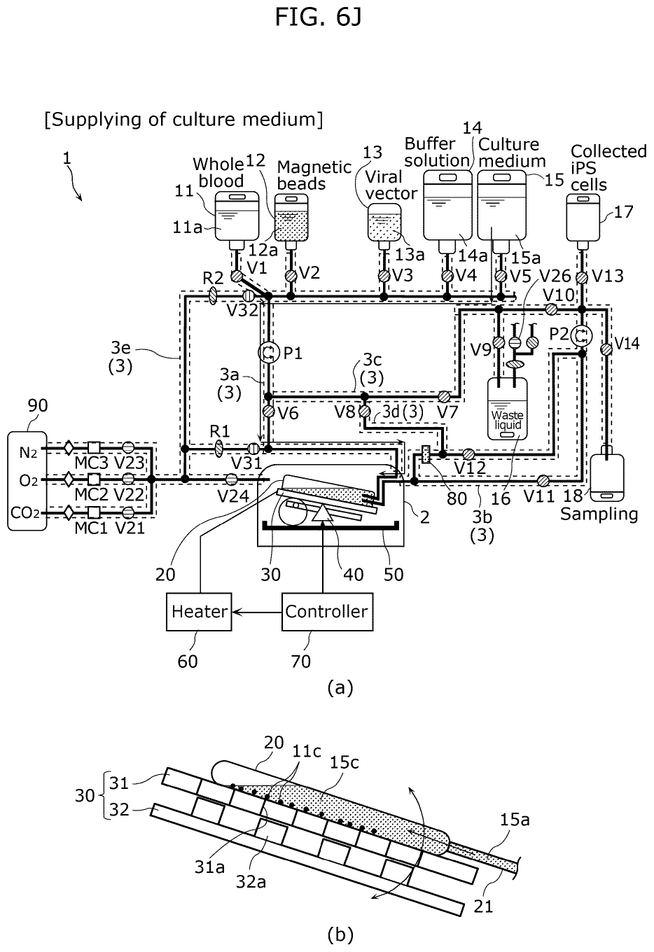

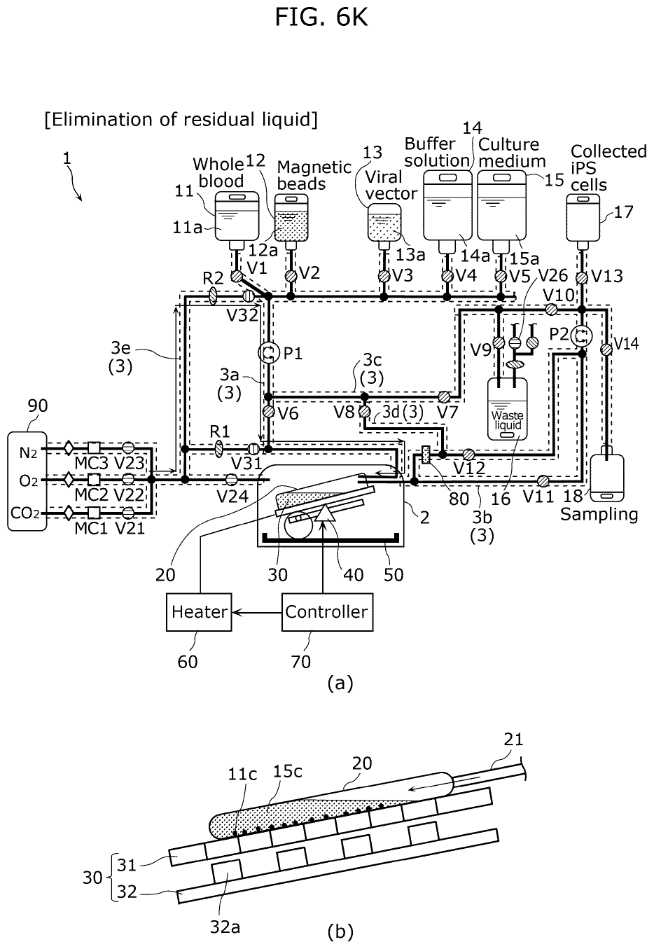

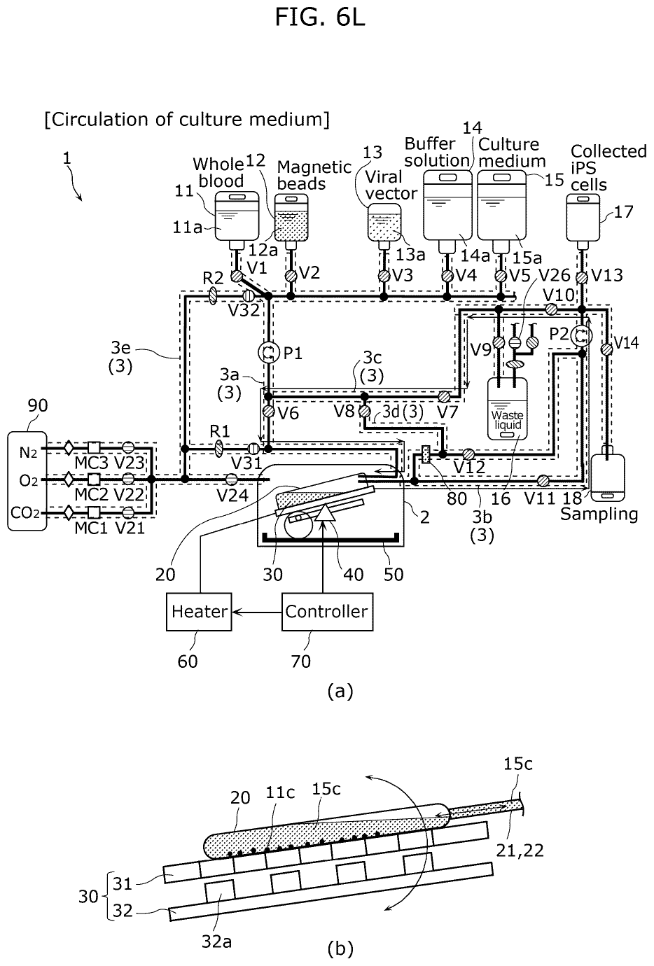

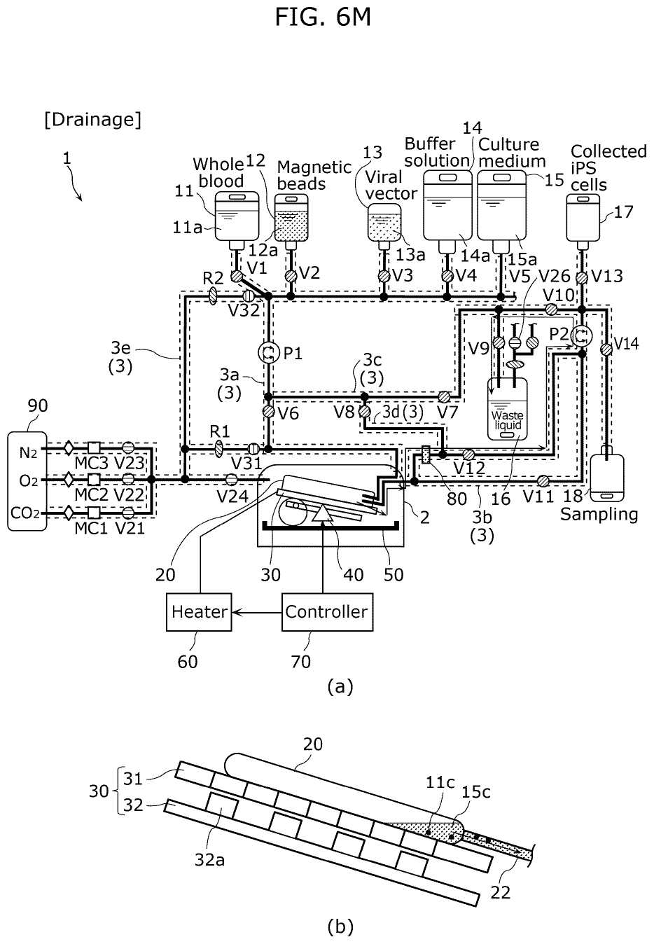

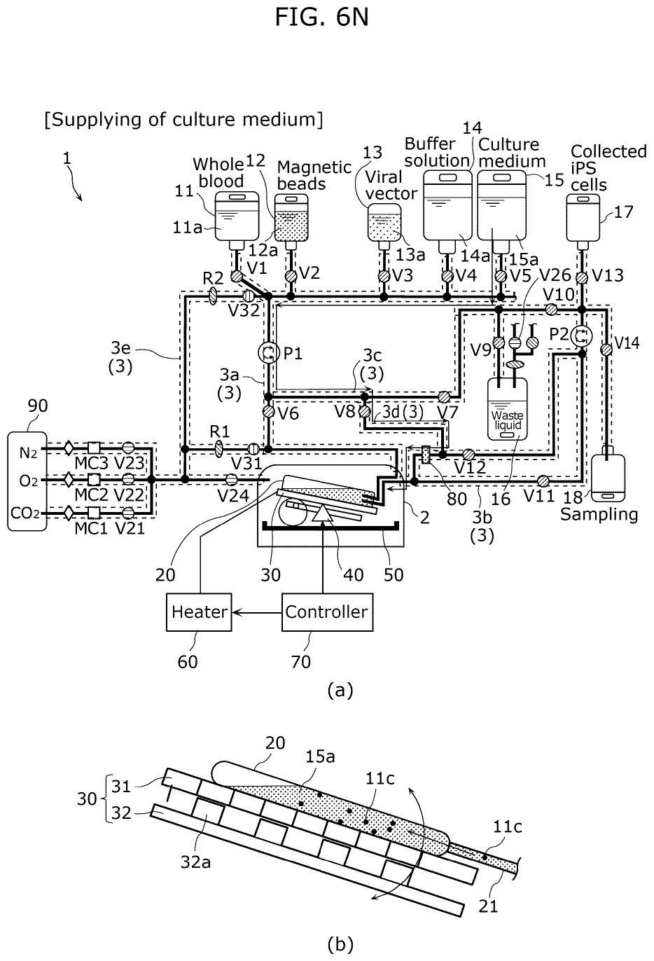

These valves, relief valves, pumps, mass controllers, and the like are controlled by controller 70. This makes it possible to automatically perform the culturing of cells consistent with a predetermined cell culture protocol, without human intervention. Note that valves, relief valves, pumps, and the like other than those illustrated in FIG. 1 may be provided to flow path 3. Next, a cell culture method in which cell culture device 1 illustrated in FIG. 1 is used will be described with reference to FIG. 6A to FIG. 60. FIG. 6A to FIG. 60 are drawings for describing a cell culture method according to an embodiment. Note that in FIG. 6A to FIG. 60, (a) illustrates the flow (the arrows in the drawings) of liquid or gas in flow path 3 of cell culture device 1, and (b) illustrates the orientation of the tilt of vessel arrangement stand 30 and the orientation of the tilt of culture vessel 20 arranged on vessel arrangement stand 30. Furthermore, in each of the following steps, the open/closed states of valves V1 to V14, valves V21 to V26, and valves V31 to V33 are suitably controlled such that liquid or gas flows to a predetermined flow path.

First, as illustrated in (a) in FIG. 6A, culture vessel 20 is set on vessel arrangement stand 30 of processing chamber 2. Furthermore, liquid holding vessels in which predetermined liquids are held, such as cell vessel 11, are set, and empty liquid holding vessels in which no liquids are held, such as waste liquid collection vessel 16, are set.

Then, as illustrated in (a) in FIG. 6A, gas is supplied to cell vessel 11, in which blood is held as first liquid 11a. Specifically, gas supply device 90 supplies air to cell vessel 11 via gas supply path 3e and supply path 3a. This makes it possible to facilitate sending all of first liquid 11a (the blood). For example, as air, mixed gas that includes nitrogen, oxygen, and carbon dioxide (CO₂ concentration of 5%) is supplied to cell vessel 11.

Note that in the supplying of the gas to cell vessel 11, as illustrated in (a) in FIG. 6A, culture vessel 20 is tilted such that the supply port 21 side (the right side in the drawings) is the lower side, but this is not intended to be limiting. In other words, culture vessel 20 need not be tilted. Furthermore, in this step, stage 31 and magnet member 32 are separated from each other, whereby vessel arrangement stand 30 is in the magnet OFF state. Thus, culture vessel 20 is arranged on stage 31, and is not in contact with magnet member 32.

Next, as illustrated in (a) in FIG. 6B, liquid that includes cells is supplied to culture vessel 20 (cell supplying step). In the present embodiment, blood that is first liquid 11a and includes whole blood stem cells is supplied, as first cells 11b, to culture vessel 20. Specifically, pump P1 causes first liquid 11a to be sent from cell vessel 11 to culture vessel 20 via supply path 3a. As illustrated in (b) in FIG. 6B, in this step, first liquid 11a is supplied to culture vessel 20 via supply port 21.

Furthermore, in this step, culture vessel 20 is in a tilted state such that the supply port 21 side (the right side in the drawings) of culture vessel 20 is the lower side. This makes it possible to gather first liquid 11a to the supply port 21 side of culture vessel 20.

Further, in this step, vessel arrangement stand 30 is oscillated by oscillation mechanism 41. For example, vessel arrangement stand 30 (culture vessel 20) is oscillated in an angular range of 10°±3° and at a speed of 0.1 reciprocations/second. Thus oscillating vessel arrangement stand 30 makes it possible to oscillate and sway culture vessel 20 arranged on vessel arrangement stand 30. This makes it possible to agitate first liquid 11a held in culture vessel 20.

Note that after first liquid 11a is supplied to culture vessel 20, gas supply device 90 may send gas through supply path 3a via gas supply path 3e to cause first liquid 11a remaining in supply path 3a to return to cell vessel 11 or be sent to culture vessel 20. This makes it possible to push out unneeded residual liquid of first liquid 11a remaining in supply path 3a to eliminate the residual liquid from supply path 3a. Consequently, in the supplying of liquid to culture vessel 20 in the next step and after, unneeded residual liquid infiltrating culture vessel 20 can be prevented. As a result, contamination due to unneeded residual liquid can be prevented from occurring. Note that the gas to be sent through supply path 3a may be a mixed gas in which gases of the three of nitrogen, oxygen, and carbon dioxide are mixed, or may be a gas that includes any one or two of nitrogen, oxygen, and carbon dioxide. In this way, not only liquid, but also gas flows through supply path 3a. In other words, supply path 3a is jointly used as a liquid supply path and a gas supply path.

Next, as illustrated in (a) in FIG. 6C, second liquid 12a that includes magnetic beads is supplied to culture vessel 20 (magnetic bead-supplying step). Specifically, pump P1 causes second liquid 12a to be sent from magnetic bead vessel 12 to culture vessel 20 via supply path 3a. As illustrated in (b) in FIG. 6C, in this step, second liquid 12a including magnetic beads 12b is supplied to culture vessel 20 via supply port 21.

In this way, second liquid 12a is supplied to culture vessel 20 in which first liquid 11a is held, whereby magnetic beads 12b are adsorbed to first cells 11b (hematopoietic stem cells) included in first liquid 11a. In other words, by this step, first liquid 11a that includes first cells 11b to which magnetic beads 12b have attached is held in culture vessel 20. In other words, inside culture vessel 20, first cells 11b included in first liquid 11a can be magnetized by magnetic beads 12b. Specifically, inside culture vessel 20, mixed liquid 12c in which first liquid 11a (blood) and second liquid 12a are mixed becomes present, and within mixed liquid 12c, first cells 11b to which magnetic beads 12b have attached become included.

Note that in the supplying of second liquid 12a to culture vessel 20, when no air is held in magnetic bead vessel 12, gas supply device 90 may supply air to magnetic bead vessel 12 via gas supply path 3e and supply path 3a. This makes it possible to enable sending all of second liquid 12a. For example, as air, a mixed gas that includes nitrogen, oxygen, and carbon dioxide (CO₂ concentration of 5%) is supplied to magnetic bead vessel 12.

Next, as illustrated in (a) in FIG. 6D, culture vessel 20 is oscillated by oscillating vessel arrangement stand 30. For example, oscillation mechanism 41 oscillates vessel arrangement stand 30 (stage 31) in an angular range smaller than the tilt angle of vessel arrangement stand 30 (stage 31) tilted with respect to the horizontal direction. As an example, when the tilt angle of vessel arrangement stand 30 (stage 31) is 10°, oscillation mechanism 41 oscillates vessel arrangement stand 30 in an angular range of 10°±5° and at a speed of 0.1 reciprocations/second, by tilting culture vessel 20 such that the supply port 21 side (the right side in the drawings) of culture vessel 20 is the upper side to gather mixed liquid 12c to the side of culture vessel 20 opposite to the supply port 21 side.

This makes it possible to agitate mixed liquid 12c that includes first liquid 11a and second liquid 12a. Thus, as illustrated in (b) in FIG. 6D, an adsorption reaction is promoted between first cells 11b (hematopoietic stem cells) and magnetic beads 12b, inside mixed liquid 12c. Furthermore, oscillating vessel arrangement stand 30 makes it possible to agitate mixed liquid 12c inside culture vessel 20; thus, first cells 11b can be mixed with air and made uniform.

Next, as illustrated in (a) and (b) in FIG. 6E, the magnet ON state is applied while culture vessel 20 is tilted. Specifically, after the oscillating of vessel arrangement stand 30 has stopped, movement mechanism 42 brings magnet member 32 closer to stage 31, without changing the tilt of vessel arrangement stand 30 (tilt angle of 10°), to apply the magnet ON state.

Thus, due to magnets 32a of magnet member 32 coming closer to culture vessel 20 arranged on vessel arrangement stand 30, a magnetic load is exerted by magnets 32a on culture vessel 20. Consequently, as illustrated in (b) in FIG. 6E, in mixed liquid 12c of culture vessel 20, first cells 11b (the hematopoietic stem cells) to which magnetic beads 12b are attached are attracted by the magnetic force of magnets 32a and drawn to the vessel arrangement stand 30 side. Thus, first cells 11b to which magnetic beads 12b are attached are gathered to the inner surface of culture vessel 20 on the vessel arrangement stand 30 side, and held at the location at which they have been attracted by magnets 32a.

Furthermore, in the step of attracting first cells 11b, vessel arrangement stand 30 (stage 31) is tilted with respect to the horizontal direction. Thus, the attraction of first cells 11b by magnets 32a begins in a state in which culture vessel 20 arranged on stage 31 is tilted. Due to thus tilting culture vessel 20, mixed liquid 12c at the end of culture vessel 20 can be collected even if mixed liquid 12c inside culture vessel 20 is present in a small amount, whereby mixed liquid 12c that includes first cells 11b can be collected to a position at which magnets 32a are present. This makes it possible to attract first cells 11b included in mixed liquid 12c by magnets 32a to easily extract first cells 11b, even if mixed liquid 12c inside culture vessel 20 is present in a small amount.

As illustrated in (a) in FIG. 6F, mixed liquid 12c is discharged from culture vessel 20 in a state of first cells 11b being attracted, by magnets 32a, to culture vessel 20 holding mixed liquid 12c that includes first cells 11b to which magnetic beads 12b are attached. In other words, the liquid inside culture vessel 20 is drained in a state in which first cells 11b are attracted by magnets 32a. Specifically, mixed liquid 12c is discharged, by pump P2, from culture vessel 20 via discharge port 22 of culture vessel 20 and discharge path 3b, and discharged mixed liquid 12c is collected in waste liquid collection vessel 16. At this time, first cells 11b that have been attracted by magnets 32a are held inside culture vessel 20 without being discharged from culture vessel 20. As shown in (b) in FIG. 6F, this makes it possible to discharge the unneeded liquid and selectively retain, inside culture vessel 20, only first cells 11b (the hematopoietic stem cells) that have been attracted by magnets 32a. In other words, discharging mixed liquid 12c makes it possible to extract first cells 11b within culture vessel 20.

Thus, by the series of steps in FIG. 6C to FIG. 6F, liquid (mixed liquid 12c) is discharged from culture vessel 20 while first cells 11b are attracted, by magnets 32a, to culture vessel 20 holding liquid (mixed liquid 12c) that includes first cells 11b to which magnetic beads 12b are attached. This makes it possible to extract first cells 11b within culture vessel 20.

Note that the step of FIG. 6F need not be the only extracting step in which first cells 11b are extracted. The extracting step of extracting first cells 11b may include, in addition to the step of FIG. 6F, the two prior steps of the step of supplying magnetic beads 12b to culture vessel 20 (the step of FIG. 6C), and the oscillating step (the step of FIG. 6D).

Furthermore, vessel arrangement stand 30 (stage 31) may be tilted when mixed liquid 12c is discharged from culture vessel 20 to extract first cells 11b. In other words, first cells 11b may be extracted while culture vessel 20 is tilted. This makes it possible to gather mixed liquid 12c to a part within culture vessel 20, even if mixed liquid 12c inside culture vessel 20 is present in a small amount. Thus, first cells 11b included in mixed liquid 12c can be easily extracted.

In the present embodiment, culture vessel 20 is in a tilted state such that the discharge port 22 side (the right side in the drawings) of culture vessel 20 is the lower side. This makes it possible to gather mixed liquid 12c to the discharge port 22 side of culture vessel 20, whereby mixed liquid 12c can be easily discharged via discharge port 22. As an example, the tilt angle of vessel arrangement stand 30 (the tilt angle of culture vessel 20) is 10°. Note that in this step, vessel arrangement stand 30 is not being oscillated.

Furthermore, in the discharging of mixed liquid 12c from culture vessel 20, first cells 11b to which magnetic beads 12b are attached may be attracted by using, among the plurality of magnets 32a of magnet member 32, at least one magnet 32a positioned on a fulcrum side (the point S side in FIG. 4) when vessel arrangement stand 30 is tilted. This makes it possible to efficiently extract first cells 11b.

Note that after mixed liquid 12c has been discharged from culture vessel 20, gas supply device 90 may send gas through discharge path 3b via gas supply path 3e to cause mixed liquid 12c remaining in discharge path 3b to be sent to waste liquid collection vessel 16. This makes it possible to eliminate, from discharge path 3b, unneeded residual liquid remaining in discharge path 3b. In this way, not only liquid, but also gas flows through discharge path 3b. In other words, discharge path 3b is jointly used as a liquid discharge path and a gas discharge path.

Next, as illustrated in (a) in FIG. 6G, buffer solution 14a is supplied to culture vessel 20 in order to clean supply path 3a and the inside of culture vessel 20. Specifically, pump P1 causes buffer solution 14a to be sent from buffer vessel 14 to culture vessel 20 via supply path 3a. As illustrated in (b) in FIG. 6G, in this step, buffer solution 14a is supplied to culture vessel 20 via supply port 21. Note that in the supplying of buffer solution 14a, the magnet ON state is active. In other words, buffer solution 14a is supplied to culture vessel 20 with magnets 32a having been brought closer to culture vessel 20.

Further, in this step, vessel arrangement stand 30 may be oscillated by oscillation mechanism 41. For example, vessel arrangement stand 30 (culture vessel 20) is oscillated in an angular range of 0°±10° and at a speed of 0.1 reciprocations/second. Thus oscillating vessel arrangement stand 30 makes it possible to oscillate and sway culture vessel 20 arranged on vessel arrangement stand 30. This makes it possible to efficiently wash the inside of culture vessel 20.

Next, as illustrated in (a) in FIG. 6H, buffer solution 14a supplied to culture vessel 20 is discharged from culture vessel 20. Specifically, pump P2 causes buffer solution 14a to be discharged from culture vessel 20 via discharge port 22 of culture vessel 20 and discharge path 3b, whereby the discharged buffer solution 14a is collected in waste liquid collection vessel 16. In the discharging of buffer solution 14a, the magnet ON state is active. Accordingly, first cells 11b are not discharged from culture vessel 20. Note that in this step, vessel arrangement stand 30 is not oscillated.

Moreover, until the inside of culture vessel 20 is thoroughly cleaned, the step of supplying buffer solution 14a (the step of FIG. 6G) and the step of draining buffer solution 14a (the step of FIG. 6H) may be alternately repeated a plurality of times.

Note that after buffer solution 14a has been discharged from culture vessel 20, gas supply device 90 may send gas through discharge path 3b to cause buffer solution 14a remaining in discharge path 3b to be sent to waste liquid collection vessel 16. This makes it possible to eliminate, from discharge path 3b, unneeded residual liquid remaining in discharge path 3b.

Next, as illustrated in (a) in FIG. 6I, third liquid 13a that includes a viral vector is supplied to culture vessel 20, in which first cells 11b that have been extracted are present (viral vector supplying step). Specifically, pump P1 causes third liquid 13a to be supplied from viral vector vessel 13 to culture vessel 20 via supply path 3a.

Thus supplying third liquid 13a that includes the viral vector to culture vessel 20 makes it possible to infect first cells 11b with a virus to generate second cells 11c. In other words, first cells 11b can be changed to second cells 11c inside culture vessel 20. In the present embodiment, iPS cells that are second cells 11c are generated from hematopoietic stem cells that are first cells 11b. Note that before supplying third liquid 13a to culture vessel 20, supply path 3a may be filled with the culture medium in advance.

Furthermore, in this step, as illustrated in (b) in FIG. 6I, culture vessel 20 is in a tilted state such that the supply port 21 side (the right side in the drawings) of culture vessel 20 is the lower side. This makes it possible to gather first liquid 11a to the supply port 21 side of culture vessel 20. As an example, the tilt angle of culture vessel 20 is 10°.

Further, in this step, vessel arrangement stand 30 may be oscillated by oscillation mechanism 41. For example, vessel arrangement stand 30 (culture vessel 20) is oscillated in an angular range of 10°±5° and at a speed of 0.1 reciprocations/second. Thus oscillating vessel arrangement stand 30 makes it possible to oscillate and sway culture vessel 20 arranged on vessel arrangement stand 30. This makes it possible to agitate third liquid 13a inside culture vessel 20, whereby the infection of first cells 11b with the virus can be promoted. This makes it possible to efficiently generate second cells 11c within culture vessel 20.

Furthermore, in this step, as illustrated in (b) in FIG. 6I, the attraction of first cells 11b by magnets 32a may be stopped. In other words, third liquid 13a may be supplied to culture vessel 20 in the magnet OFF state. This makes it possible for first cells 11b to move freely within third liquid 13a, whereby the virus infection of first cells 11b can be further promoted. For example, the magnet OFF state may be applied when the cleaning of the inside of culture vessel 20 by buffer solution 14a ends. Specifically, magnet member 32 is moved by movement mechanism 42 such that magnets 32a become farther away from stage 31.

Note that after third liquid 13a has been supplied to culture vessel 20, gas supply device 90 may send gas through supply path 3a via gas supply path 3e to cause third liquid 13a remaining in supply path 3a to return to viral vector vessel 13 or be sent to culture vessel 20. This makes it possible to push out unneeded residual liquid that remains in supply path 3a to eliminate the unneeded residual liquid from supply path 3a. Note that the gas to be sent through supply path 3a may be a mixed gas in which gases of the three of nitrogen, oxygen, and carbon dioxide are mixed, or may be a gas that includes only one or two of nitrogen, oxygen, and carbon dioxide. Next, as illustrated in (a) in FIG. 6J, culture medium 15a is supplied to culture vessel 20 (culture medium-supplying step). In other words, culture medium 15a is supplied to culture vessel 20 holding third liquid 13a that includes second cells 11c. Specifically, pump P1 causes culture medium 15a to be supplied from culture medium vessel 15 to culture vessel 20 via supply path 3a. As illustrated in (b) in FIG. 6J, culture medium 15a is supplied to culture vessel 20 via supply port 21.

In this step, culture medium 15a is supplied to culture vessel 20 holding third liquid 13a that includes second cells 11c, whereby third liquid 13a and culture medium 15a are mixed inside culture vessel 20. In other words, inside culture vessel 20, culture medium 15c including third liquid 13a comes to be present, as a mixed liquid. Moreover, second cells 11c are included in culture medium 15c. Accordingly, second cells 11c inside culture vessel 20 undergo cell division and propagate.

Thus supplying culture medium 15a to culture vessel 20 makes it possible to culture, within culture vessel 20, second cells 11c generated inside culture vessel 20 from the extracted first cells 11b. In other words, the extraction of first cells 11b from which second cells 11c are generated, and the culturing of second cells 11c generated from first cells 11b can be performed in the same single culture vessel 20. In the present embodiment, the extraction of hematopoietic stem cells from blood and the culturing of iPS cells, which are the target cells and have been generated from the hematopoietic stem cells, are performed within the same single culture vessel 20.

The supply of culture medium 15a to culture vessel 20 may be performed in accordance with the cell division cycle of second cells 11c. In this case, culture vessel 20 may be tilted and the tilt may be gradually reduced, in accordance with the fluid volume of culture medium 15a in culture vessel 20. In other words, the tilt of vessel arrangement stand 30 may be gradually reduced such that culture vessel 20 approaches a flat state. This makes it possible to, in the culturing of second cells 11c, perform optimal culturing in accordance with the fluid volume of culture medium 15a in culture vessel 20. Note that in the final stage, culture vessel 20 may be flat.

The tilt of culture vessel 20 may be controlled in accordance with the weight of culture medium 15a in culture vessel 20, detected by weight sensor 50. This makes it possible to perform optimal culturing in accordance with the fluid volume of culture medium 15a in culture vessel 20.

Furthermore, in this step, culture vessel 20 is in a tilted state such that the supply port 21 side (the right side in the drawings) of culture vessel 20 is the lower side. This makes it possible to collect culture medium 15a to the supply port 21 side of culture vessel 20. This makes it possible to efficiently culture second cells 11c, even if, when culturing second cells 11c, culture medium 15a inside culture vessel 20 is present in a small amount. As an example, the tilt angle of culture vessel 20 is 10°.

Further, in this step, vessel arrangement stand 30 may be oscillated by oscillation mechanism 41. For example, oscillation mechanism 41 may oscillate vessel arrangement stand 30 (stage 31) in an angular range smaller than the tilt angle of vessel arrangement stand 30 (stage 31) tilted with respect to the horizontal direction. As an example, when the tilt angle of vessel arrangement stand 30 (stage 31) is 10°, vessel arrangement stand 30 (culture vessel 20) is oscillated in an angular range of 10°±5° and at a speed of 0.1 reciprocations/second. Thus oscillating vessel arrangement stand 30 makes it possible to oscillate and sway culture vessel 20 arranged on vessel arrangement stand 30. This makes it possible to shake and agitate culture medium 15a inside culture vessel 20, whereby second cells 11c included in culture medium 15a can be mixed with air and made uniform. This makes it possible to more efficiently culture second cells 11c.

Furthermore, in the step of culturing second cells 11c, heater 60 may be turned on to keep the temperature of culture medium 15c in culture vessel 20 at a constant temperature (for example, 37° C.). In this case, the output of heater 60 may be controlled in accordance with the weight of the liquid inside culture vessel 20, detected by weight sensor 50, or the tilt of stage 31. This makes it possible to adjust the temperature of culture medium 15c inside culture vessel 20 to the optimal temperature.

Note that when performing heating by heater 60, as illustrated in (b) in FIG. 6J, magnet member 32 may be moved away from stage 31. In other words, in the step of culturing second cells 11c, the magnet OFF state may be applied.

Next, as illustrated in (a) in FIG. 6K, gas supply device 90 sends gas through supply path 3a via gas supply path 3e to cause culture medium 15a remaining in supply path 3a to be sent to culture vessel 20. The gas to be sent through supply path 3a may be a mixed gas in which gases of the three of nitrogen, oxygen, and carbon dioxide are mixed, or may be a gas that includes any one or two of nitrogen, oxygen, and carbon dioxide.

Thus sending the gas through supply path 3a makes it possible to send a predetermined fluid volume of culture medium 15a to culture vessel 20 by pushing out culture medium 15a remaining in supply path 3a. Furthermore, unneeded culture medium 15a remaining in supply path 3a can be eliminated.

Note that culture medium 15a remaining in supply path 3a may be not pushed into culture vessel 20, but returned to culture medium vessel 15. In this case as well, unneeded culture medium 15a remaining in supply path 3a can be eliminated.

Next, as illustrated in (a) in FIG. 6L, culture medium 15a supplied to culture medium vessel 15 is circulated (circulating step). This step of circulating culture medium 15a is included in the culturing step. Specifically, culture medium 15c is circulated by pump P2 such that culture medium 15c in culture vessel 20 passes through discharge path 3b, connection path 3c, and supply path 3a, in this order, and returns to culture vessel 20. This makes it possible, in the culturing of second cells 11c inside culture vessel 20, to agitate and mix culture medium 15c by circulating culture medium 15c such that culture medium 15c passes inside and outside of culture vessel 20, using supply path 3a, discharge path 3b, and connection path 3c. This makes it possible to uniformly culture second cells 11c. Note that in (a) in FIG. 6L, in the discharging of culture medium 15c that is inside culture vessel 20, culture medium 15c passes through discharge path 3b (a first discharge path), to which filter 80 is not provided.

As illustrated in (b) in FIG. 6L, in this step, culture medium 15c is circulated by discharging culture medium 15c that is inside culture vessel 20 from discharge port 22, and supplying the discharged culture medium 15c to culture vessel 20 from supply port 21.

Furthermore, in the circulating of culture medium 15c, culture vessel 20 may be in a tilted state. In other words, culture medium 15c may be circulated in a state in which stage 31 on which culture vessel 20 is arranged is tilted. For example, as illustrated in (b) in FIG. 6L, culture vessel 20 is put into a tilted state such that the supply port 21 side (the right side in the drawings) of culture vessel 20 is the upper side. As an example, the tilt angle of culture vessel 20 is 30°. Thus circulating culture medium 15c with culture vessel 20 in the tilted state makes it possible to efficiently circulate culture medium 15c, even if culture medium 15c inside culture vessel 20 is present in a small amount.

Furthermore, in the circulating of culture medium 15c, vessel arrangement stand 30 may be oscillated by oscillation mechanism 41. For example, the oscillation of vessel arrangement stand 30 (culture vessel 20) is performed for one minute every 30 minutes in an angular range of 30°±16° and at a speed of 0.05 reciprocations/second, and this is performed four times. Thus oscillating vessel arrangement stand 30 makes it possible to oscillate and sway culture vessel 20 arranged on vessel arrangement stand 30. This makes it possible to circulate culture medium 15a while agitating culture medium 15a, whereby culture medium 15c can be efficiently mixed. This makes it possible to efficiently generate second cells 11c.

As culture medium 15c is circulated and time passes, culture medium 15c degenerates due to, e.g., metabolites secreted from second cells 11c. Thus, culture medium 15c may be replaced with a new culture medium at an appropriate time during the culture period.

In this case, as illustrated in (a) in FIG. 6M, first, culture medium 15c is drained. Specifically, pump P2 causes culture medium 15c to be discharged from culture vessel 20 via discharge path 3b, whereby the discharged culture medium 15c is collected in waste liquid collection vessel 16. At this time, culture medium 15c includes second cells 11c, which serve as the targets, but it is necessary to prevent second cells 11c from being disposed of in waste collection vessel 16. Accordingly, in the present embodiment, in the discharging of culture medium 15c that is inside culture vessel 20, culture medium 15c is made to pass through discharge path 3b (the second discharge path), to which filter 80 is provided. Filter 80 traps second cells 11c included in culture medium 15c that is discharged from culture vessel 20. In this way, discharging culture medium 15c that is inside culture vessel 20 causes second cells 11c included in culture medium 15c to be trapped by filter 80.

Note that when draining culture medium 15c, culture vessel 20 is in the tilted state. Specifically, as illustrated in (b) in FIG. 6M, culture vessel 20 is in a state of being tilted such that the discharge port 22 side of culture vessel 20 (the right side in the drawings) is the lower side.

Next, as illustrated in (a) in FIG. 6N, new culture medium 15a is supplied to culture vessel 20. At this time, it is necessary to return second cells 11c, having been trapped by filter 80, to culture vessel 20. Accordingly, in the present embodiment, culture medium 15a is supplied to culture vessel 20 via a dedicated culture medium supply path 3d for supplying culture medium 15a to culture vessel 20. Specifically, pump P1 causes the new culture medium 15a supplied from culture medium vessel 15 to be supplied to culture vessel 20 by going through supply path 3a, connection path 3c, and culture medium supply path 3d, in this order. Due to culture medium 15a thus being supplied to culture medium supply path 3d, second cells 11c trapped by filter 80 are returned to culture vessel 20.

In this way, in the present embodiment, when culture medium 15c that includes second cells 11c is discharged from culture vessel 20, second cells 11c are trapped by filter 80, and in the supplying of the new culture medium 15a to culture vessel 20 during culture medium replacement, second cells 11c trapped by filter 80 are supplied, together with culture medium 15a, to culture vessel 20. This makes it possible to perform culturing using the new culture medium 15a, without disposing of second cells 11c. In other words, the culturing of second cells 11c can be continued. Furthermore, thus replacing the old culture medium 15c with the new culture medium 15a makes it possible to efficiently culture second cells 11c, whereby the propagation time of second cells 11c can be shortened.

Note that when supplying the new culture medium 15a, culture vessel 20 is in the tilted state. For example, as illustrated in (b) in FIG. 6N, culture vessel 20 is in a tilted state such that the supply port 21 side (the right side in the drawings) of culture vessel 20 is the lower side, but this is not intended to be limiting.

Furthermore, culture medium replacement processing (the step of FIG. 6M and the step of FIG. 6N) may be performed not once, but two or more times.

Furthermore, after the culture medium replacement processing, gas supply device 90 may send gas through supply path 3a, connection path 3c, and culture medium supply path 3d to cause culture medium 15a remaining in supply path 3a, connection path 3c, and culture medium supply path 3d to return to culture medium vessel 15 or be sent to culture vessel 20. This makes it possible to eliminate the unneeded residual liquid from supply path 3a by pushing out the unneeded residual liquid of culture medium 15a that remains in supply path 3a, connection path 3c, and culture medium supply path 3d. Further, gas supply device 90 may send gas through discharge path 3b via gas supply path 3e to cause culture medium 15a remaining in discharge path 3b to be sent to waste liquid collection vessel 16. This makes it possible to eliminate the residual liquid from discharge path 3b.

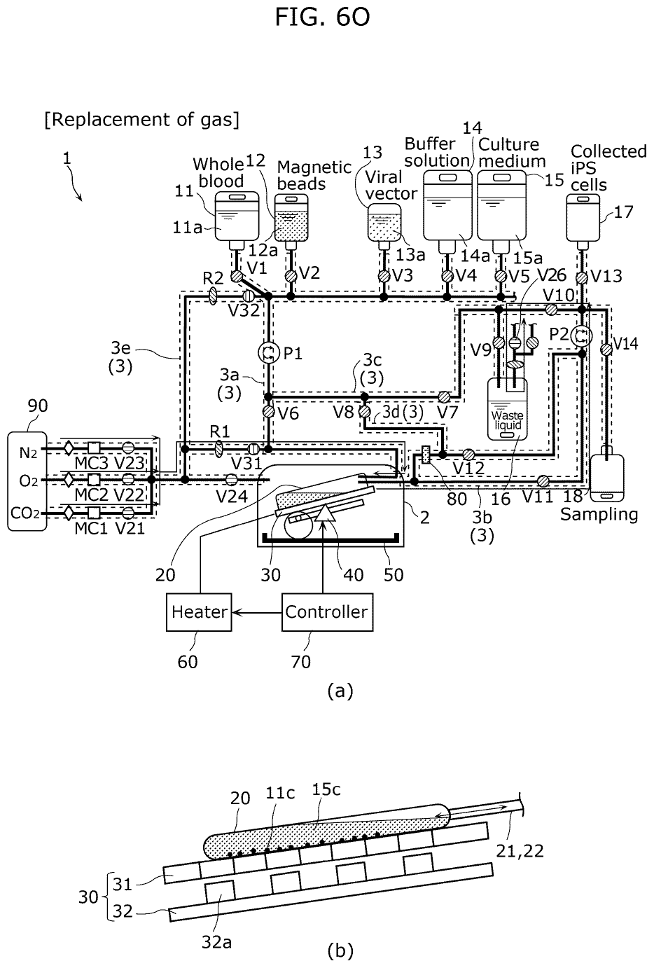

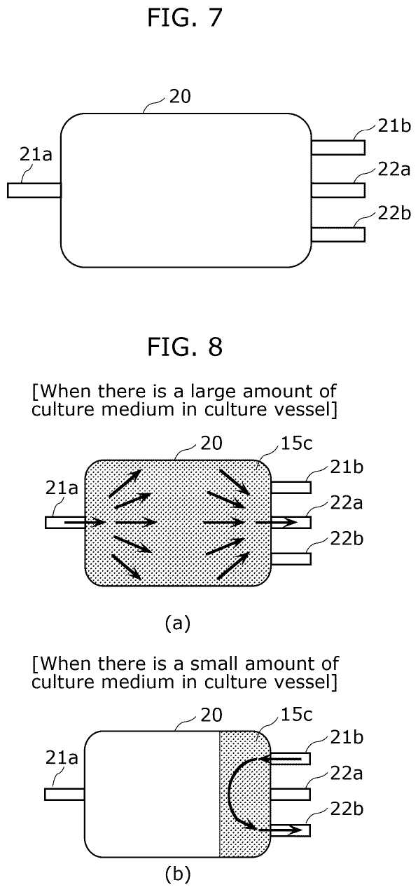

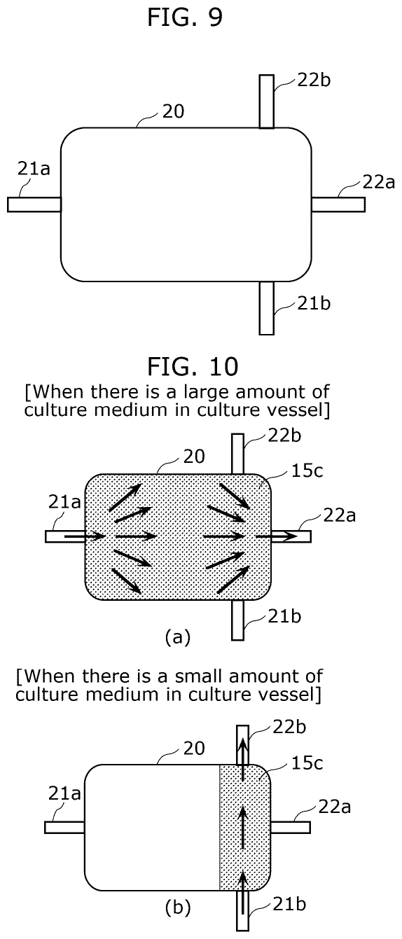

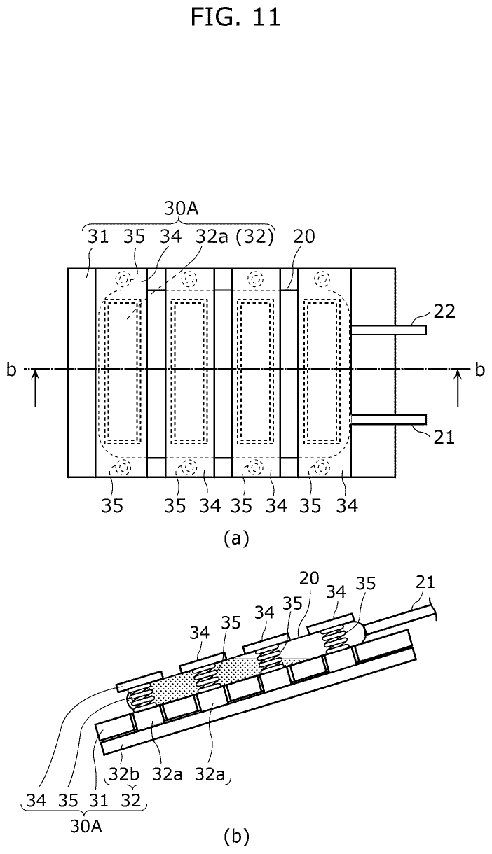

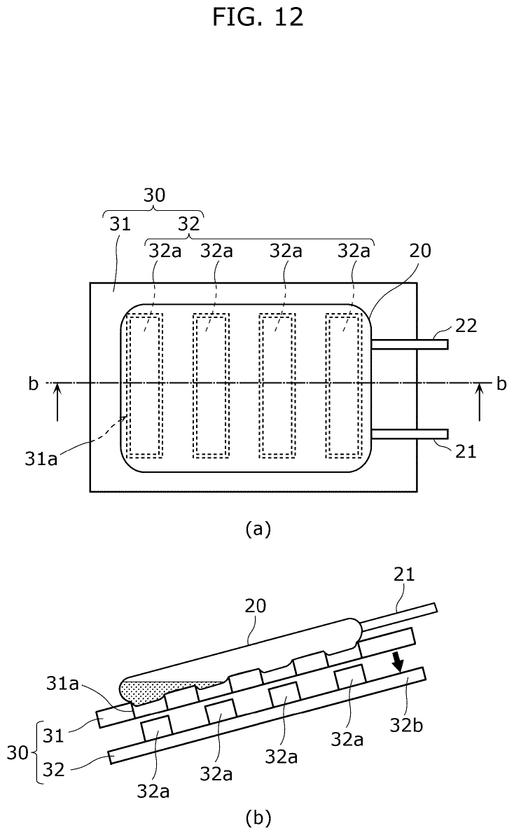

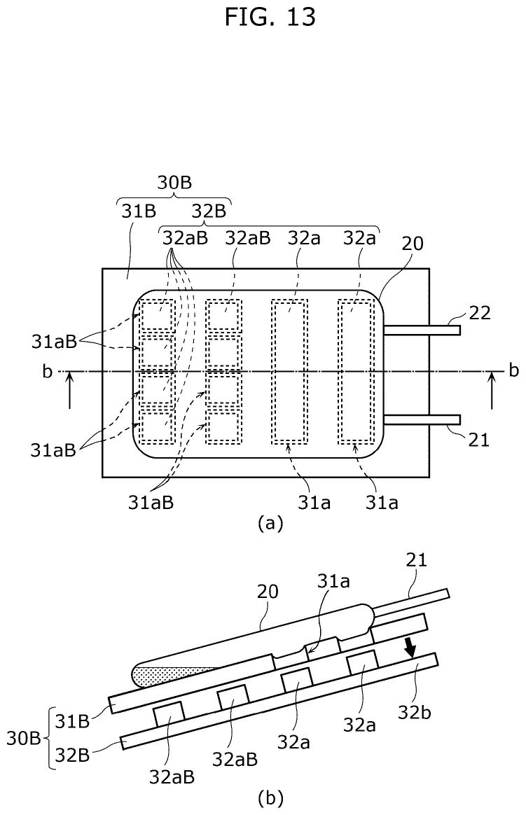

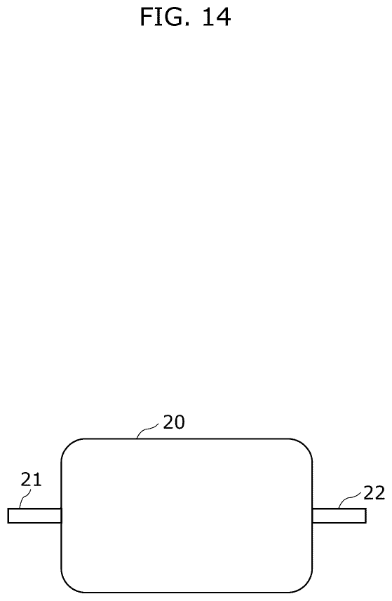

Furthermore, in the above-described step of culturing second cells 11c, the gas inside culture vessel 20 may be replaced. Specifically, as illustrated in (a) of FIG. 60, in the culturing of second cells 11c using culture vessel 20, gas supply device 90 supplies gas to supply path 3a via gas supply path 3e to cause the gas to be supplied to culture vessel 20. At this time, in the present embodiment, gas supply path 3e is directly connected to culture vessel 20 as well as supply path 3a, whereby the gas can be efficiently supplied to culture vessel 20.

Furthermore, in the present embodiment, one end of discharge path 3b, which serves as a gas discharge path, is directly connected to culture vessel 20, and the other end of discharge path 3b, which serves as a gas discharge path, is open via waste liquid collection vessel 16. This makes it possible to push out the old gas that is inside culture vessel 20, by using the gas that is newly supplied to culture vessel 20. In other words, the gas inside culture vessel 20 can be easily replaced.

Thus replacing the gas inside culture vessel 20 makes it possible to make the culture atmosphere inside culture vessel 20, during the culturing of second cells 11c, an optimal environment for culturing. Thus, it is possible to more efficiently culture second cells 11c.

In this case, the gas supplied to culture vessel 20 may be, similarly to air, a mixed gas including nitrogen, oxygen, and carbon dioxide (for example, a CO₂ concentration of 5%). This makes it possible to maintain the culture atmosphere inside culture vessel 20, during the culturing of second cells 11c, to be an optimal environment similar to that inside of human tissue. This makes it possible to efficiently culture second cells 11c.

Note that gas supply path 3e branches into an upper side with pump P1, and a lower side, but in the gas replacement, as illustrated in (a) in FIG. 60, the gas may be supplied to culture vessel 20 via gas supply path 3e on the lower side.

Furthermore, as illustrated in (b) in FIG. 60, in the gas replacement, the gas may be supplied to culture vessel 20 in a state of culture vessel 20 being tilted. This makes it possible to separate the liquid (culture medium 15c) and the gas inside culture vessel 20, whereby the gas replacement can be performed without passing the gas through the liquid inside culture vessel 20.

Furthermore, in the gas replacement in the step of culturing second cells 11c, heater 60 heats culture vessel 20 arranged on stage 31. At this time, the output of heater 60 is controlled in accordance with the temperature measured by temperature sensor 33. Furthermore, the output of heater 60 may be controlled in accordance with the amount of gas supplied to gas supply path 3e and the temperature measured by temperature sensor 33. In other words, the output of heater 60 may be controlled in accordance with not only the temperature, but also the amount of gas. This makes it possible to adjust the culture atmosphere inside culture vessel 20 arranged on stage 31 to be an environment suitable for culturing.

Furthermore, while not illustrated, in the gas replacement, the gas to be newly supplied may be heated by a heater for gas. The heater for gas may be provided to a site within gas supply path 3e, or may be provided to gas supply device 90. By thus heating the gas to be newly supplied, the gas replacement can be performed such that the temperature of the gas inside culture vessel 20 is an optimal temperature. This makes it possible to make the culture atmosphere inside culture vessel 20, during the culturing of second cells 11c, an optimal environment for culturing. Thus, it is possible to efficiently culture second cells 11c.

In the above manner, second cells 11c that are the target cells can be cultured and propagated. Subsequently, the cultured second cells 11c are collected in cell collection vessel 17. In this case, second cells 11c may be collected by attracting second cells 11c by Furthermore, at this time, the plurality of using magnets 32a. magnets 32a may be controlled such that the cultured second cells 11c are attracted by using a greater number of the plurality of magnets 32a than the at least one magnet 32a. Moreover, the cultured second cells 11c may be attracted by using all of the plurality of magnets 32a. This makes it possible, even when a large quantity of second cells 11c have been propagated by the culturing, to easily attract the large quantity of second cells 11c by using magnets 32a. Thus, it is possible to efficiently collect second cells 11c.

As described above, the cell culture method according to the present embodiment includes: extracting, in culture vessel 20 holding first liquid 11a that includes first cells 11b to which magnetic beads 12b are attached, first cells 11b by discharging first liquid 11a from culture vessel 20 while first cells 11b are attracted to culture vessel 20 by magnets 32a; and culturing, in culture vessel 20, second cells 11c by supplying culture medium 15a to culture vessel 20, second cells 11c having been generated in culture vessel 20 from first cells 11b extracted.

Thus, in the cell culture method according to the present embodiment, first cells 11b from which second cells 11c are generated are extracted in culture vessel 20 that is for culturing second cells 11c that serve as the targets. In other words, the step of extracting first cells 11b from first liquid 11a, and the step of culturing second cells 11c obtained from first cells 11b are performed using the same culture vessel 20. This makes it possible to efficiently culture second cells 11c that serve as the targets.

Furthermore, a first aspect of the cell culture device according to the present embodiment includes: cell vessel 11 holding first liquid 11a that includes first cells 11b; culture medium vessel 15 holding culture medium 15a; and culture vessel 20 holding culture medium 15a for culturing second cells 11c. Furthermore, cell culture device 1: extracts, in culture vessel 20 holding first liquid 11a that includes first cells 11b to which magnetic beads 12b are attached, first cells 11b by discharging first liquid 11a from culture vessel 20 while first cells 11b are attracted to culture vessel 20 by magnets 32a; and cultures, in culture vessel 20, second cells 11c generated in culture vessel 20 from first cells 11b extracted.

Thus, in cell culture device 1 according to the present disclosure, first cells 11b from which second cells 11c are generated are extracted using culture vessel 20 in which second cells 11c that serve as the targets are cultured. In other words, the extraction of first cells 11b from first liquid 11a, and the culturing of second cells 11c obtained from first cells 11b are performed using the same culture vessel 20. This makes it possible to efficiently culture second cells 11c that serve as the targets.

Furthermore, a second aspect of the cell culture device according to the present embodiment includes: cell vessel 11 holding first liquid 11a that includes first cells 11b; culture medium vessel 15 holding culture medium 15a; culture vessel 20 for culturing second cells 11c generated from first cells 11b; supply path 3a for supplying first liquid 11a and culture medium 15a to culture vessel 20; and discharge path 3b for discharging vessel liquid present in culture vessel 20. Furthermore, supply path 3a and discharge path 3b are provided separately from each other. Furthermore, in culture vessel 20 as well, supply port 21 connected to supply path 3a and discharge port 22 connected to discharge path 3b are provided separately from each other.

Due to this configuration, supply path 3a for supplying first liquid 11a and culture medium 15a to culture vessel 20, and discharge path 3b for discharging the vessel liquid present in culture vessel 20 are provided as separate flow paths. Furthermore, in culture vessel 20 as well, supply port 21 for supplying first liquid 11a and culture medium 15a to culture vessel 20, and discharge port 22 for discharging the vessel liquid present in culture vessel 20 are provided as separate pumps. This makes it possible to inhibit the time period when discharging the liquid from culture vessel 20 (the time of liquid discharge) from becoming longer. Specifically, in the extracting of first cells 11b, the time period required for discharging the mixed liquid of first liquid 11a and second liquid 12a from culture vessel 20 can be inhibited from becoming longer, and the time period required for discharging culture medium 15c from culture vessel 20 for, e.g., culture medium replacement can be inhibited from becoming longer.

Moreover, separating supply path 3a and discharge path 3b makes it unnecessary to use supply path 3a at the time of liquid discharge. Furthermore, in culture vessel 20 as well, separating supply port 21 and discharge port 22 makes it unnecessary to use supply port 21 at the time of liquid discharge. Consequently, unneeded residual liquid at the time of liquid discharge no longer remains in supply path 3a. Thus, in the supplying of the next liquid to culture vessel 20 by using supply path 3a, the unneeded residual liquid also being supplied to culture vessel 20 can be prevented. Consequently, contamination due to unneeded residual liquid can be prevented from occurring.

Further, due to separating supply path 3a and discharge path 3b, the flow path of each of supply path 3a and discharge path 3b can be designed separately. In other words, an optimal flow path can be designed for each of supply path 3a and discharge path 3b. Specifically, it is possible to: design the flow path diameter, etc. of supply path 3a or discharge path 3b in accordance with the flow amount of the liquid that flows through supply path 3a or the liquid that is discharged from discharge path 3b; design the flow path diameter taking into account only the liquid that flows through supply path 3a; and the like This makes it possible to supply liquids such as first liquid 11a and culture medium 15a to the culture vessel in an appropriate amount.

These result in making it possible, due to separating supply path 3a and discharge path 3b, to efficiently culture second cells 11c that serve as the targets, even in the case of closed-type cell culture device 1.

Furthermore, a third aspect of the cell culture device according to the present embodiment includes: stage 31 on which culture vessel 20 for culturing second cells 11c generated from first cells 11b is arranged; and oscillation mechanism 41 that oscillates stage 31.

Due to this configuration, it is possible to tilt culture vessel 20 by changing the tilt of stage 31 on which culture vessel 20 holding culture medium 15a is arranged. This makes it possible to gather culture medium 15a to a part within culture vessel 20, whereby it is possible to efficiently culture second cells 11c serving as the targets, even if, during the culturing of second cells 11c, culture medium 15a inside culture vessel 20 is present in a small amount. Furthermore, it is possible to appropriately change the tilt of stage 31 for another purpose. For example, the tilt of stage 31 can be changed by a tilt angle suitable for each case among: a case of supplying liquid to culture vessel 20; a case of discharging liquid that is inside culture vessel 20; and a case of replacing gas that is inside culture vessel 20.

Furthermore, culture vessel 20 can be oscillated by oscillating stage 31 on which culture vessel 20 is arranged. This makes it possible to agitate the liquid inside culture vessel 20. This makes it possible to, for example, efficiently extract first cells 11b from first liquid 11a in the extracting, and efficiently culture second cells 11c in the culturing.

Furthermore, when oscillating stage 31 by using oscillation mechanism 41, the oscillation fulcrum (point S) when oscillating stage 31 may be positioned closer to, among supply path 3a and discharge path 3b, the path that has more flow path routes.

When stage 31 is oscillated, flow path 3 connected to culture vessel 20 receives damage; however, by providing the oscillation fulcrum closer to, among supply path 3a and discharge path 3b in flow path 3, the path that has more flow path routes, the damage as an overall flow path can be lessened. This makes it possible to realize cell culture device 1 having high reliability.

Furthermore, a fourth aspect of the cell culture device according to the present embodiment includes: vessel arrangement stand 30 on which culture vessel 20 for culturing second cells 11c generated from first cells 11b is arranged. Moreover, vessel arrangement stand 30 includes: stage 31 on which culture vessel 20 is arranged; and magnets 32a, wherein before culturing second cells 11c, first liquid 11a that includes first cells 11b to which magnetic beads 12b are attached is held in culture vessel 20.

Thus providing magnets 32a to stage 31 on which culture vessel 20 is arranged makes it possible to extract first cells 11b from which second cells 11c are generated, in culture vessel 20 for culturing second cells 11c. In other words, the step of extracting first cells 11b from first liquid 11a, and the step of culturing second cells 11c obtained from first cells 11b can be performed using the same culture vessel 20. This makes it possible to efficiently culture second cells 11c that serve as the targets, even in the case of closed-type cell culture device 1.

Furthermore, a fifth aspect of the cell culture device according to the present embodiment includes: gas supply path 3e connected to culture vessel 20; and gas supply device 90 that supplies gas to gas supply path 3e. Specifically, gas supply path 3e is directly connected to culture vessel 20.

This configuration makes it possible to directly supply gas to culture vessel 20 by using gas supply device 90. This makes it possible to maintain the atmosphere within culture vessel 20 as a suitable environment by promptly supplying gas to culture vessel 20 in accordance with changes in the air composition within culture vessel 20. For example, the culture atmosphere within culture vessel 20 during the culturing of second cells 11c can be made into an optimal environment for culturing. Thus, second cells 11c serving as the targets can be efficiently cultured.

Note that in the above-described embodiment, one supply port 21 and one discharge port 22 are provided to culture vessel 20, but this is not intended to be limiting.

For example, as illustrated in FIG. 7, the two supply ports of first supply port 21a and second supply port 21b, and the two discharge ports of first discharge port 22a and second discharge port 22b may be provided to culture vessel 20. Each of first supply port 21a, second supply port 21b, first discharge port 22a, and second discharge port 22b are provided separately. Specifically, in FIG. 7, first supply port 21a is provided to one of the two short sides of the vessel main body of culture vessel 20, and first discharge port 22a, second supply port 21b, and second discharge port 22b are provided to the other of the two short sides of the vessel main body of culture vessel 20.

This configuration makes it possible, when circulating the culture medium inside and outside of culture vessel 20 by using supply path 3a, which serves as the collection route, and discharge path 3b, to select a circulation route for culture medium 15c in accordance with the fluid volume of culture medium 15c in culture vessel 20.

For example, as illustrated in (a) in FIG. 8, when there is a large amount of culture medium 15c in culture vessel 20, culture medium 15c in culture vessel 20 may be circulated by using first supply port 21a and first discharge port 22a.

On the other hand, as illustrated in (b) in FIG. 8, when there is a small amount of culture medium 15c in culture vessel 20, culture medium 15c in culture vessel 20 may be circulated by using second supply port 21b and second discharge port 22b.

Thus changing the combinations of supply path 3a (the collection path) and discharge path 3b in accordance with the fluid volume of culture medium 15c in culture vessel 20 makes it possible to circulate culture medium 15c in a flow that follows the long side of culture medium 15c. This makes it possible to efficiently perform the circulation of culture medium 15c.

Furthermore, when two supply ports and two discharge ports are provided to culture vessel 20, the configuration illustrated in FIG. 7 is not intended to be limiting. Specifically, as illustrated in FIG. 9, first supply port 21a may be provided to one of the two short sides of the vessel main body of culture vessel 20, first discharge port 22a may be provided to the other of the two short sides of the vessel main body of culture vessel 20, and second supply port 21b and second discharge port 22b may be separately provided to one side and the other side, respectively, of the two long sides of the vessel main body of culture vessel 20.

This configuration as well makes it possible, when circulating the culture medium inside and outside of culture vessel 20 by using supply path 3a, which serves as the collection route, and discharge path 3b, to select a circulation route for culture medium 15c in accordance with the fluid volume of culture medium 15c in culture vessel 20.

For example, as illustrated in (a) in FIG. 10, when there is a large amount of culture medium 15c in culture vessel 20, culture medium 15c in culture vessel 20 may be circulated by using first supply port 21a and first discharge port 22a.

On the other hand, as illustrated in (b) in FIG. 10, when there is a small amount of culture medium 15c in culture vessel 20, culture medium 15c in culture vessel 20 may be circulated by using second supply port 21b and second discharge port 22b.

In this case as well, it is possible to circulate culture medium 15c in a flow that follows the long side of culture medium 15c, in accordance with the fluid volume of culture medium 15c in culture vessel 20. This makes it possible to efficiently perform the circulation of culture medium 15c.

Note that when there is a large amount of culture medium 15c in culture vessel 20 (the case of (a) in FIG. 8 and (a) in FIG. 10), the circulation of culture medium 15c may be performed by using not only first supply port 21a and first discharge port 22a, but also second supply port 21b and second discharge port 22b.

Furthermore, in the above-described embodiment, the outside of the upper side of culture vessel 20 arranged on stage 31 was open, but this is not intended to be limiting. For example, as illustrated in FIG. 11, culture vessel 20 arranged on stage 31 may be held down on stage 31 by hold-down plates 34. FIG. 11 is a diagram illustrating a first variation of vessel arrangement stand 30A. In FIG. 11, (a) is a top view, and (b) is a cross-sectional view of (a) when cut along the b-b line.

Specifically, as illustrated in FIG. 11, vessel arrangement stand 30A according to the present variation has stage 31, magnet member 32, and hold-down plates 34. Hold-down plates 34 have the function of holding down, on stage 31, culture vessel 20 arranged on stage 31. In the present variation, vessel arrangement stand 30A has four hold-down plates 34 aligned in a stripe pattern with gaps therebetween. The whole of culture vessel 20 is held down by these four hold-down plates 34.

Thus providing hold-down plates 34 makes it possible to uniformly hold down culture vessel 20 on stage 31, whereby second cells 11c can be more efficiently cultured. Furthermore, since it is also possible to uniformly hold down culture vessel 20 on magnets 32a, the attraction of first cells 11b by magnets 32a can be performed effectively.

Moreover, as illustrated in FIG. 11, vessel arrangement stand 30A further has elastic bodies 35, and hold-down plates 34 are held by elastic bodies 35. Elastic bodies 35 are, for example, springs, and are respectively interposed between each hold-down plate 34 and stage 31.

This configuration makes it possible to hold culture vessel 20 on stage 31 in accordance with the bulging of culture vessel 20. Specifically, the bulging of culture vessel 20 arranged on stage 31 changes in accordance with the amount of liquid supplied to culture vessel 20. In this case, as in the present variation, the height positions of hold-down plates 34 are automatically adjusted in accordance with the bulging of culture vessel 20 due to hold-down plates 34 being held by elastic bodies 35. This makes it possible to hold culture vessel 20 on stage 31 in accordance with the bulging of culture vessel 20. Thus, hold-down plates 34 excessively holding down culture vessel 20 and causing damage to culture vessel 20 itself or to the liquid inside culture vessel 20 can be inhibited.

The plurality of elastic bodies 35 illustrated in FIG. 11 are disposed around culture vessel 20 arranged on stage 31. Furthermore, in the example illustrated in FIG. 11, one pair of elastic bodies 35 are disposed, one on each of both sides, in the long direction of one magnet 32a, and four columns are aligned, each column consisting of this one magnet 32a and one pair of elastic bodies 35.

Furthermore, in the present variation, hold-down plates 34 are provided in positions that oppose magnets 32a. This configuration makes it possible to more effectively attract first cells 11b by magnets 32a.

Furthermore, in the present variation, hold-down plates 34 are constituted from a magnetic material. Due to this configuration, each hold-down plate 34 functions as a magnetic core, whereby the magnetic flux density passing through culture vessel 20 can be improved. This makes it possible to more effectively attract first cells 11b by magnets 32a.

Furthermore, as illustrated in FIG. 12, in the above-described embodiment, magnet member 32 is moved away from stage 31 at times such as while performing heating by using heater 60. In this case, culture vessel 20 enters through holes 31a of stage 31. Consequently, it may not be possible to appropriately heat the liquid at the portions of culture vessel 20 that have entered through holes 31a, it may not be possible to perform appropriate mixing, and/or the like. Note that FIG. 12 is a diagram illustrating the configuration of vessel arrangement stand 30 used in the above-described embodiment illustrated in FIG. 3, and an example of culture vessel 20 arranged on vessel arrangement stand 30. In FIG. 12, (a) is a top view, and (b) is a cross-sectional view of (a) when cut along the b-b line.

Accordingly, as in vessel arrangement stand 30B illustrated in FIG. 13, with respect to vessel arrangement stand 30 of FIG. 12, through hole 31a at a portion corresponding to one column in stage 31 may be divided into a plurality, and a plurality of through holes 31aB may be provided to a portion corresponding to one column in stage 31B. Furthermore, magnet 32a at a portion corresponding to one column in magnet member 32 may be divided into a plurality, and a plurality of magnets 32aB may be provided to a portion corresponding to one column in magnet member 32B. In FIG. 13, two columns of through holes 31aB and magnets 32aB are each divided into four. Note that FIG. 13 is a diagram illustrating the configuration of vessel arrangement stand 30B when culture vessel 20 has been arranged thereon. In FIG. 13, (a) is a top view, and (b) is a cross-sectional view of (a) when cut along the b-b line.

This configuration makes it possible to reduce the size of through holes 31aB of stage 31B, and of magnets 32aB inserted into through holes 31aB. This makes it possible, as illustrated in (b) in FIG. 13, to inhibit a part of culture vessel 20 from entering through holes 31aB, even if magnet member 32B is moved away from stage 31B at a time such as while performing heating by using heater 60. Thus, it is possible to, e.g., appropriately heat and appropriately mix the liquid in culture vessel 20. Note that as illustrated in FIG. 13, only some of the columns of the through holes and magnets, among the plurality of columns, may be divided, rather than dividing all of the through holes and magnets among the through holes and magnets of the plurality of columns. In this case, the through holes and the magnets at sites close to the portion located on the lower side of culture vessel 20 when culture vessel 20 is tilted may be divided.

### Variations

Although the cell culture device and the cell culture method according to the present disclosure have been described based on the embodiments, the present disclosure is not limited to the above-described embodiments.

For example, in the above-described embodiment, supply port 21 and discharge port 22 provided to culture vessel 20 were provided to the same side of culture vessel 20, but this is not intended to be limiting. In other words, supply port 21 and discharge port 22 provided to culture vessel 20 may be provided to different sides of culture vessel 20. In this case, as illustrated in FIG. 14, supply port 21 and discharge port 22 may be provided to opposing positions to interpose culture vessel 20. For example, in FIG. 14, supply port 21 is provided to one short side of two opposing short sides of culture vessel 20, and discharge port 22 is provided to the other short side of the two opposing short sides of culture vessel 20.

Due to this configuration, supply port 21 directly connected to gas supply path 3e, and discharge port 22 directly connected to one end of discharge path 3b, which is a gas discharge path, are provided at positions that oppose each other to interpose culture vessel 20. This makes it possible to perform gas replacement without passing gas within the liquid inside culture vessel 20, by tilting culture vessel 20 such that discharge port 22 is on the upper side and supply port 21 is on the lower side.

Furthermore, in the above-described embodiment, the iPS cells were produced from hematopoietic stem cells by using a viral vector, but this is not intended to be limiting. In other words, the iPS cells may be produced from hematopoietic stem cells without using a viral vector. In this case, in the above-described embodiment, the step of supplying, to culture vessel 20, third liquid 13a that includes the viral vector is unneeded.

Furthermore, in the above-described embodiment, in the supplying of the gas to culture vessel 20, the gas is supplied to culture vessel 20 such that the gas does not pass through the liquid held in culture vessel 20, but this is not intended to be limiting. In other words, the gas may be supplied to culture vessel 20 such that the gas passes through the liquid held in culture vessel 20. Specifically, the gas may be supplied to culture vessel 20 while tilting culture vessel 20 such that supply port 21 of culture vessel 20 is on the lower side. For example, when the liquid held in culture vessel 20 is a culture medium, the gas can be supplied to culture vessel 20 such that a gas including carbon dioxide passes through the culture medium. This makes it possible to incorporate the carbon dioxide into the culture medium.

Furthermore, as described above, in the above-described embodiment, the various vessels used in cell culture device 1 are replaceable. For example, cell vessel 11, magnetic bead vessel 12, viral vector vessel 13, buffer vessel 14, culture medium vessel 15, waste liquid collection vessel 16, cell collection vessel 17, and sampling vessel 18, as well as culture vessel 20, are replaceable vessels. Thus, in the above-described embodiment, these vessels were constituent elements of cell culture device 1, but these vessels need not be constituent elements of cell culture device 1. Furthermore, as described above, in the above-described embodiment, flow path 3 in cell culture device 1 is replaceable. For example, supply path 3a, discharge path 3b, connection path 3c, culture medium supply path 3d, and gas supply path 3e are replaceable flow paths. Thus, in the above-described embodiment, these flow paths were constituent elements of cell culture device 1, but these flow paths need not be constituent elements of cell culture device 1. Note that these flow paths may each be a part of the above-described various vessels. Thus, when replacing the various vessels, these flow paths may also be replaced. For example, when replacing culture vessel 20, flow path 3 that is connected to culture vessel 20 may also be replaced.

Furthermore, in the above-described embodiment, cell culture device 1 was of a closed type and the cell culture method was a closed system, but this is not intended to be limiting. The techniques of the present disclosure may be applied to an open-type cell culture device, and may be applied to an open-system cell culture method. However, the techniques of the present disclosure are suited to a closed-type cell culture device and a closed-system cell culture method.

The present disclosure also includes other forms obtained by making various modifications to the above embodiments that can be conceived by those skilled in the art, as well as forms obtained by combining constituent elements and functions of the embodiments as desired, within a scope not departing from the spirit of the present disclosure.

### INDUSTRIAL APPLICABILITY

The techniques of the present disclosure are useful as a cell culture method, a cell culture device, a vessel to be used in a cell culture device, and the like for culturing cells such as iPS cells.

1. A cell culture device comprising:
a vessel arrangement stand on which a culture vessel is arranged, the culture vessel being a vessel in which a second cell generated from a first cell is cultured, wherein
the vessel arrangement stand includes:
a stage on which the culture vessel is arranged; and
a magnet, and
before culturing the second cell, a liquid that includes the first cell to which a magnetic particle is attached is held in the culture vessel.

2. The cell culture device according to claim 1, comprising:
a pump that discharges a liquid from the culture vessel, wherein
when the pump discharges, from the culture vessel, the liquid that includes the first cell to which the magnetic particle is attached, the magnet applies a magnetic load to the culture vessel to attract the first cell.

3. The cell culture device according to claim 1, comprising:
an oscillation mechanism that oscillates the stage to cause the stage to tilt with respect to a horizontal direction, wherein
the vessel arrangement stand includes a plurality of magnets each of which is the magnet.

4. The cell culture device according to claim 3, wherein
the plurality of magnets are disposed at a plurality of positions in a direction in which the stage tilts.

5. The cell culture device according to claim 3, comprising:
a pump that discharges a liquid from the culture vessel, wherein
when the pump discharges, from the culture vessel, the liquid that includes the first cell to which the magnetic particle is attached, the first cell to which the magnetic particle is attached is attracted by using, among the plurality of magnets, at least one magnet that is positioned closer to a fulcrum at which the stage tilts.

6. The cell culture device according to claim 5, wherein
the plurality of magnets are controlled to attract the second cell by using a greater number of the plurality of magnets than the at least one magnet, the second cell having been cultured.

7. The cell culture device according to claim 1, wherein
a heater is provided to the stage.

8. The cell culture device according to claim 7, wherein
the magnet and the heater are alternately arranged.

9. The cell culture device according to claim 8, wherein
the magnet and the heater are arranged in a stripe pattern.

10. The cell culture device according to claim 7, comprising:
a controller that controls output of the heater in accordance with a weight of a liquid in the culture vessel or a tilt of the stage.

11. The cell culture device according to claim 1, comprising:
a hold-down plate that holds down, on the stage, the culture vessel arranged on the stage.

12. The cell culture device according to claim 11, wherein
the hold-down plate is held by an elastic body.

13. The cell culture device according to claim 11, wherein
the hold-down plate is provided at a position that opposes the magnet.

14. The cell culture device according to claim 13, wherein the hold-down plate is constituted from a magnetic material.

15. The cell culture device according to claim 1, wherein
the magnet is a permanent magnet, and
the cell culture device comprises a movement mechanism that is capable of moving the magnet to change a distance between the magnet and the stage.

16. The cell culture device according to claim 1, wherein
a culture medium and the liquid that includes the first cell are supplied to the culture vessel, the culture medium being for culturing the second cell.

17. The cell culture device according to claim 16, wherein
a supply path and a discharge path are connected to the culture vessel, the supply path being for supplying the culture medium and the liquid that includes the first cell to the culture vessel, the discharge path being for discharging vessel liquid present in the culture vessel,
the liquid that includes the first cell is supplied to the culture vessel from a cell vessel,
the culture medium is supplied to the culture vessel from a culture medium vessel, and
the cell vessel, the culture medium vessel, and the culture vessel define a closed space when the cell vessel and the culture medium vessel are each connected to the supply path and the discharge path.

18. The cell culture device according to claim 1, wherein
a liquid including a viral vector is supplied to the culture vessel to infect the first cell with a virus to generate the second cell.

19. The cell culture device according to claim 1, wherein
the first cell is a hematopoietic stem cell,
the liquid that includes the first cell is blood, and
the second cell is an induced pluripotent stem (IPS) cell.

20. A culture vessel arranged in the cell culture device according to claim 1.

21. The culture vessel according to claim 20, comprising:
a supply port through which a culture medium and the liquid that includes the first cell are supplied to the culture vessel, the culture medium being from a culture medium vessel, the liquid that includes the first cell being from a cell vessel; and
a discharge port through which vessel liquid present in the culture vessel is discharged, wherein
the supply port and the discharge port are provided separately from each other.

22. The culture vessel according to claim 21, wherein
the cell vessel, the culture medium vessel, and the culture vessel define a closed space when the cell vessel and the culture medium vessel are connected to the supply port.

23. The culture vessel according to claim 21, wherein
in a top view, a shape of the culture vessel includes two short sides that oppose each other in one direction, and two long sides that oppose each other in an other direction that intersects the one direction,
the culture vessel includes: a first supply port and a second supply port that are each the supply port; and a first discharge port and a second discharge port that are each the discharge port,
the first supply port is provided to one of the two short sides of the culture vessel,
the first discharge port is provided to an other of the two short sides of the culture vessel, and
the second supply port and the second discharge port are provided to the other of the two short sides of the culture vessel, or are separately provided to one side and an other side, respectively, of the two long sides of the culture vessel.

24. The culture vessel according to claim 21, wherein
a supply path is connected to the supply port, the supply path being for supplying, to the culture vessel, the culture medium and the liquid that includes the first cell,
a discharge path is connected to the discharge port, the discharge path being for discharging the vessel liquid present in the culture vessel, and
the supply path and the discharge path are provided separately from each other.

25. The culture vessel according to claim 24, wherein
a cross-sectional area of a flow path of the supply path and a cross-sectional area of a flow path of the discharge path are different from each other.

26. The culture vessel according to claim 24, wherein
a filter that traps the second cell is provided to the discharge path, the second cell being included in the culture medium discharged from the culture vessel,
the supply path includes a culture medium supply path through which the culture medium is supplied to the culture vessel, and
the culture medium is supplied to the culture medium supply path to cause the second cell trapped by the filter to return to the culture vessel.
